(19) United States
(12) Patent Application Publication
(10) Pub. No.: US 2007/0275037 A1
(43) Pub. Date: Nov. 29, 2007

(54) Recombinant vitellogenin enriched feed

(76) Inventors: Jeak Ling Ding, Singapore (SG); Eng Hwa Lim, Persiaran Raja Chulan (MY); Toong Jin Lam, Singapore (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/534,657

(22) PCT Filed: Nov. 11, 2003
(86) PCT No.: PCT/SG03/00266

§ 371 Date: Jan. 27, 2006

Related U.S. Application Data
(60) Provisional application No. 60/425,263, filed on Nov. 12, 2002.

(51) Int. Cl. C12N 1/18; C12P 7/64; C12N 15/74

(52) U.S. Cl. 424/442; 435/134; 435/254.23; 435/484

(57) ABSTRACT

The invention provides an expression vector for expression of recombinant vitellogenin in an eukaryotic host. An eukaryotic host, including yeast comprising the expression vector according to the invention may be used as a feed or feed additive for both oviparous and non-oviparous animals, including domesticated animals. A transgenic yeast according to the invention contain increased levels of essential amino acids and fatty acids and may be used as a direct feed or fed to an intermediate live feed such as rotifers or artemias to increase the survival rates of oviparous animal or broodstock.

### CROSS REFERENCE TO RELATED APPLICATIONS

This application claims the benefit of U.S. Provisional Application Ser. No. 60/425,263 filed Nov. 12, 2002, the contents of which is hereby incorporated by reference.

### FIELD OF INVENTION

The invention relates to feed technology and to recombinant technology and in particular relates to feed enriched with recombinant vitellogenin. Embodied in this invention is the use of live recombinants to enrich live intermediate live feed and the development of microdiets for larvae.

### BACKGROUND OF INVENTION

Vitellogenesis, the formation of yolk proteins, has been extensively studied. Yolk proteins are derived from a large lipophosphoglycoprotein precursor, vitellogenin (Vtg). Vtg is synthesized in the liver under estrogen influence, extensively modified post-translationally, secreted into the bloodstream and sequestered by the oocytes via specific Vtg receptors (Lim et al., 1991; Li et al., 2003). After internalization, Vtg is cleaved into subunits of yolk proteins, lipovitellin I (LVI), phosvitin, and lipovitellin II (LVII). Vtg serves to provide a pool of amino acids for the embryos, and also functions as a carrier protein for phosphates, lipids, carbohydrates, ions (eg. Ca²⁺, Mg²⁺, Zn²⁺), vitamins and possibly, hormones (Sappington and Raikhel, 1998). These essential nutrients amassed in the yolk provide exclusive nourishment to the developing embryos of all oviparous animals. For most fish, the newly-hatched larvae continue to derive nutrients from the yolk sac until first feeding. Hence, proper formation and deposition of the yolk proteins in the oocyte and the ability of the embryos to utilize the yolk reserves are crucial for reproductive success.

Broodstock nutrition is a crucial factor influencing larval quality and survival. In particular, lipids have been shown to affect the spawning and the egg quality of many fish species (Sivaloganathan et al., 1998). Deficiency of n-3 polyunsaturated fatty acids (PUFA), in broodstock has been linked to reduced fecundity, and lower rates of fertilization and hatching in many species. Thorough evaluation of lipids as a source of energy at the embryonic and larval stages in relation to proteins and carbohydrates indicates that lipids play a significant role in the early life stages of marine larvae (Rainuzzo et al., 1997).

Considerable research has been conducted on the nutritional requirements of commercially produced animals. One important problem the aquaculture industry encounters is high mortality at the early stage of larval development presumably due to poor egg quality, small size and simple digestive systems at the start of feeding. One of the keys to enhancement of biological performance is through feed technology. To maximize feed utilization, diets targeted for specific developmental stages of aquaculture species, and high protein live feed including yeast, rotifer, and brine shrimp have been formulated and tested (Fitzsimmons et al., 1999; Lochmann, 2001; Su et al., 1997). However, rotifers for instance, showed batch inconsistencies in nutritional value depending on their primary feed (Su et al., 1994). With new bioreactor technology yeast enriched by recombinant technology has become a cost-effective protein for energy source (Chen et al., 2000).

There remains however a need for improved feed for oviparous larvae and broodstock, in particular to increase the survival rate of larvae.

### SUMMARY OF THE INVENTION

Yolk proteins represent the major nutrient constituent in the yolk. The present invention is based in part on the recognition that oviparous larval mortality and oviparous broodstock egg quality may be improved by providing a feed enriched with yolk proteins. Since yeast is a low-cost micronutrient, we set out to construct a yeast strain for synthesis of single cell protein, actively producing the precursor of yolk proteins. Without being limited to any particular theory, it is believed that such transgenic host expressing recombinant vitellogenin proteins may partially mimic the natural yolk formation process and thereby provide an exogenous source of nutrients for larvae and broodstock particularly beneficial for optimal oviparous larval survival rates and/or oviparous broodstock egg quality.

In accordance with one aspect of the present invention, there is provided a recombinant expression vector wherein the expression vector comprises a vitellogenin gene operably linked to a promoter, wherein the promoter is functional in a eukaryotic host suitable for use as a feed or a feed additive.

In accordance with another aspect of the present invention, there is provided a transgenic eukaryotic host suitable for use as a feed or feed additive, such as, for example, yeast wherein the eukaryotic host comprises an expression vector according to various embodiments of the invention.

In a specific embodiment, the transgenic yeast according to the invention intracellularly expresses a recombinant vitellogenin.

In accordance with yet another aspect of the invention, there is provided a method for increasing the level of polyunsaturated fatty acids in a transgenic host according to the invention, including a transgenic yeast, comprising culturing the transgenic host in media comprising fish oil.

In accordance with yet another aspect of the invention, there is provided a method for increasing the survival rates of oviparous larvae, and a method for increasing broodstock egg quality of an oviparous animal comprising the step of feeding to the larvae or to the broodstock a transgenic host, or an intermediate live feed that has been fed a transgenic host, including a transgenic yeast according to different embodiments of the invention.

In accordance with yet another aspect of the invention, there is provided a method for enriching an intermediate live feed comprising the step of feeding to the intermediate live feed a transgenic host, such as a transgenic yeast according to various embodiments of the invention.

In a specific embodiment, the intermediate live feed is Artemia nauplii.

In another aspect of the invention, there is provided use of recombinant vitellogenin for delivery of a therapeutic material such as hormones, vitamins, minerals, ions and nucleic acid to the maternal oocytes of an oviparous animal.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

FIG. 1 is a schematic representation of the rVtg expression vectors: Vtg(-SS)/pGAPZA, Vtg(VtgSS)/pGAPZA and Vtg(αSS)/pGAPZA.

FIG. 2A shows a Southern blot of genomic DNA of the transgenic P. pastoris clones indicated using a DIG-labelleled O. aureus Vtg fragment. The hybridization signal for HIS4 is to control for differences in loading amounts. The numbers below the blot indicate the copy number of the Vtg expression cassettes based on the ratio of the densitometric intensity of the Vtg bands to those of the His4.

FIG. 2B is a Western blot of vitellogenin with an apparent molecular weight of 194 kD from the lysate and membrane fractions of transgenic yeast clones indicated.

FIG. 3 depicts the effects of media composition on the expression of the recombinant vitellogenin protein.

FIG. 4 is a graph depicting recombinant vitellogenin expression as a function of culture conditions.

FIG. 5A is a graph depicting the increase in total amino acid content for two transgenic P. pastoris clones grown in shake flasks relative to a native P. pastoris control. The inset of this figure shows the increase in total amino acid content for transgenic clone #6 grown in different culture conditions. FIG. 5B is a graph showing the amino acid content of the listed amino acids of trans genic P. pastoris clone #6 relative to a native P. pastoris control.

FIG. 6 is a graph depicting the relative amount of the indicated fatty acids for transgenic P. pastoris clone #6, grown in the presence and absence of fish oil, relative to a native P. pastoris control. The inset to the graph depicts the increase in total lipid content for transgenic P. pastoris clones #6 and #52, relative to native P. pastoris (SMD1186H).

FIG. 7 shows the levels of 18:3 (n-3), 20:5 (n-3) and 22:6 (n-3) in total lipids from Artemia larvae fed 48 hr with baker's yeast SC (Saccromyces crevisiae), SMD (Pichia pastoris), recombinant yeast clone #6 (#6) or recombinant yeast clone #6 cultured in 5% cod liver oil (#6 5% oil). Data are mean ±SE of triplicate sample.

FIG. 8 shows the length, weight and survival of O. mossambicus larvae fed different types or combinations of yeast for 20 days. (A) Length of larvae fed 20 days with different types or combinations of yeast. (B). Weight of larvae fed 20 days with different types or combinations of yeast. (C) Survival of larvae fed 20 days with different types or combinations of yeast. Data are mean ±SE of triplicate sample.

FIG. 9 shows the length, weight and survival of O. mossambicus larvae fed 10 days with different types or combinations of yeast and another 10 days with Artemia (wild type). (A) Length of larvae fed 10 days with different types or combinations of yeast and another 10 days with Artemia (wild type). (B) Weight of larvae fed 10 days with different types or combinations of yeast and another 10 days with Artemia (wild type). (C) Survival of larvae fed 10 days with different types or combinations of yeast and another 10 days with Artemia (wild type). Data are mean ±SE of triplicate sample.

FIG. 10 shows the length, weight and survival of O. mossambicus larvae fed 10 days with different types of yeast and another 10 days with enriched Artemia. (A) Length of larvae fed 10 days with different types of yeast and another 10 days with enriched Artemia. (B) Weight of larvae fed 10 days with different types of yeast and another 10 days with enriched Artemia. (C) Survival of larvae fed 10 days with different types of yeast and another 10 days with enriched Artemia. Data are mean ±SE of triplicate sample.

### DETAILED DESCRIPTION OF EMBODIMENTS OF THE INVENTION

Developing larvae require three macronutrients: proteins, fats, and carbohydrates, along with many elements classified as micronutrients. Some are indispensable during fertilization as well as larval growth. Vitellogenin is one of the most crucial factors for the reproductive success and survival of oviparous embryos.

We present here a novel strategy for genetically engineering a eukaryotic host that produces recombinant Vtg (rVtg), for application as an enriched feed. In an illustrative embodiment, the host is Pichia pastoris. Pichia pastoris has been employed as a high protein feed for some domestic animals (Chen et al., 2000) and may advantageously be used to express foreign proteins as it: (1) has the ability to grow to high cell densities using inexpensive simple, defined media; (2) has the capacity to express high levels of recombinant proteins when multiple copies of the expression cassette are integrated; and (3) is competent in carrying out eukaryotic post-translational modifications (Clare et al., 1991a; 1991b; Cregg et al., 1993; Romanos, 1995).

In various aspects, the invention therefore relates to transgenic eukaryotic hosts suitable for use as a feed or feed additive that express recombinant Vtg (rVtg). The transgenic hosts in some embodiments are suitable as a feed or feed additive for an oviparous animal, and may be fed to an oviparous animal, preferably to an oviparous larvae, thereby providing an exogenous source of vitellogenin. Owing to the high nutritional value of vitellogenin, a phospholipoglycoprotein, suitable eukaryotic hosts expressing a recombinant vitellogenin may also be used as a high quality feed additive for non-oviparous animals, including domesticated animals.

In one aspect, the invention provides an expression vector that comprises a vitellogenin gene operably linked to a promoter functional in such hosts. As will be understood by those skilled in the art, the expression vector may be linear or circular. The expression vector may be single stranded or preferably double stranded. Preferably, the expression vector is a plasmid. The expression vector may integrate into a host cell chromosome or may contain an appropriate origin of replication, allowing for extra-chromosomal replication and propagation. The expression vector will often contain, in addition to a vitellogenin gene and a eukaryotic promoter, other sequences to facilitate vector propagation and selection in other host cells, such as E. coli. For example, the vector may include sequences conferring resistance to antibiotics, for example, Zeocin. In addition, the vectors of the present invention may comprise a sequence of nucleotides for one or more restriction endonuclease recognition sites.

The term “vitellogenin gene” refers to any nucleotide sequence that encodes a vitellogenin protein or a functional equivalent thereof. As used herein, “vitellogenin gene” includes DNA sequences encoding for either a mature vitellogenin protein or a precursor, for example, a sequence that also encodes a secretion signal sequence (SS). Vitellogenin gene and protein sequences are known and publicly available, for example from GenBank database and include the sequences shown in SEQ ID NOS: 1 to 20. The term “functional equivalent” is used to describe structurally and functionally related amino acids sequence that may differ from the parent amino acid by one or more deletions, substitutions, modifications or additions that do not affect the normal biological function of vitellogenin. In one embodiment, the functional equivalent will be substantially homologous, meaning that there is a substantial correspondence between the amino acid sequence of the equivalent and the parent amino acid sequence. In specific embodiments, the functional equivalent will be at least about 50%, 75%, 90% and 95% homologous.

Homology is measured using sequence analysis software such as Sequence Analysis Software Package of the Genetics Computer Group, University of Wisconsin Biotechnology Center, 1710 University Avenue, Madison, Wis. 53705. Amino acid sequences are aligned to maximize identity. Gaps may be artificially introduced into the sequence to attain proper alignment. Once the optimal alignment has been set up, the degree of homology is established by recording all of the positions in which the amino acids of both sequences are identical, relative to the total number of positions.

In one embodiment, the functional equivalent differs by one or more conservative amino acid substitutions. Conservative amino acid substitutions are substitutions among amino acids of the same class. These classes include, for example, amino acids having uncharged polar side chains, such as asparagine, glutamine, serine, threonine, and tyrosine; amino acids having basic side chains, such as lysine, arginine, and histidine; amino acids having acidic side chains, such as aspartic acid and glutamic acid; and amino acids having nonpolar side chains, such as glycine, alanine, valine, leucine, isoleucine, proline, phenylalanine, methionine, tryptophan, and cysteine. Generally, the functional equivalent will include one or more deletions, substitutions, modifications or additions in non-conserved sequences. Homology among sequences from different species may be analyzed to determine conserved sequences using, as an example, the BLAST homology searching algorithm of Altschul et al., Nucleic Acids Res.; 25:3389-3402 (1997). Vitellogenin genes from various organisms are functionally and structurally conserved and an alignment of a number of vitellogenin sequences have identified five well-aligned subdomains (Lim et al., 2001). One skilled in the art therefore can identify DNA sequences encoding functional polypeptide variants of naturally occurring vitellogenins by alignment to one or more of these subdomains.

The term “promoter” as will be understood by those skilled in the art refers to a nucleic acid sequence capable of driving the transcription of a gene to which the promoter is operably linked and a promoter is functional in a host if it is so capable in the host. In the expression vector, the vitellogenin gene is operably linked to a promoter, meaning the promoter drives the transcription of the vitellogenin gene in a host in which the promoter is functional.

The promoter may be an inducible promoter, or preferably a constitutive promoter. The term “constitutive promoter” refers to a sequence that directs transcription of the operably linked gene under most normal cellular conditions. Preferably the promoter is a strong constitutive promoter, in that it directs high levels of expression of the transgenic vitellogenin gene in the selected host cell. A number of appropriate promoters would be known to a person skilled in the art, and include the algal chloroplast atpA or rbcL promoters the yeast 3-phosphoglycerate kinase, glyceraldehyde-3-phosphate dehydrogenase (GAP), or alcohol dehydrogenase promoters or alcohol oxidase (AOX) promoter

The expression vector of the present invention may be constructed by standard techniques known to someone skilled in the art and described, for example in Sambrook et al., in Molecular Cloning: A Laboratory Manual 3^{rd} Edition, Cold Spring Harbour, Laboratory Press and other laboratory manuals. Nucleic acid molecules may be chemically synthesized using techniques such as disclosed, for example in Itakura et al U.S. Pat. No. 4,598,049; Caruthers et al U.S. Pat. No. 4,458,066; and Itakura et al U.S. Pat. Nos. 4,401,796 and 4,373,071.

Nucleic acids molecules may also be isolated and combined. By isolated it is meant that the isolated substance has been substantially separated or purified away from other components, such as biological components, with which it would otherwise be associated, for example in vivo, so that the isolated substance may itself be manipulated or processed. The term isolated therefore includes substances purified by standard purification methods, as well as substance purified by recombinant expression in a host, as well as chemically synthesized substances. A variety of strategies are available for combining and ligating individual nucleic acid molecules, and depending on the nature or the termini of the nucleic acids to be ligated, a suitable strategy will readily be apparent to one skilled in the art. As will be apparent to a person skilled in the art of molecular biology, the DNA fragments must be ligated in the proper frame to ensure that the resulting gene encodes for the desired amino acid sequence.

An expression vector of the present invention may be introduced into a host cell, which may include a cell capable of transcribing and translating the gene of the expression vector. Accordingly, the invention also provides host cells containing an expression vector of the invention and recombinant vitellogenin expressed by such host cells, which recombinant protein may be isolated and purified using well-known techniques. The term “host cell” refers not only to the particular subject cell but to the progeny or potential progeny of such a cell. Because certain modifications may occur in succeeding generations due to either cellular differentiation, mutation or environmental influences, such progeny may not, in fact, be identical to the parent cell, but are still included within the scope of the term as used herein.

Vector DNA can be introduced into cells by conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells are well known in the art and can for example be found in Sambrook et al. (Molecular Cloning: A Laboratory Manual, 3rd Edition, Cold Spring Harbor Laboratory press (2001)), and other laboratory manuals.

A cell, tissue, organ, or organism into which has been introduced a foreign nucleic acid, is considered “transformed”, “transfected”, or “transgenic”. A transgenic or transformed cell or organism also includes progeny of the cell or organism and progeny produced from a breeding program employing a transgenic organism as a parent and exhibiting an altered phenotype resulting from the presence of a recombinant nucleic acid. A transgenic organism is therefore an organism that has been transformed with a heterologous nucleic acid, or the progeny of such an organism that includes the transgene.

A eukaryotic host cell suitable for use as a feed or feed additive for an oviparous animal such as fish, into which has been introduced the expression vector according to the invention is one important aspect of this invention. Preferably, the host contains an expression vector that is stably maintained within the host cell either by autonomous replication within the host cell or by integrating into the host cell's genome. If the expression vector is an integrative vector, the vector may integrate into a random chromosomal location or, preferably, may be targeted to a specific chromosomal locus through the process of homologous recombination. In one embodiment, multiple copies of the expression vectors have integrated into the genome of the host.

The vitellogenin protein is preferably expressed intracellularly in the host. Intracellular expression as that term is used herein is intended to broadly describe the expressed vitellogenin associated with the host cell for example where the expressed protein is localized within the cell or associated with the cell membrane. In contrast, vitellogenin that is secreted into the culture media is not expressed intracellularly.

In one embodiment, the transgenic eukaryotic host according to the invention is capable of effecting post-translational modification of a eukaryotic protein such as Vtg. In one embodiment, the eukaryotic cell may be cultured to a significant cell density. Ideally, the eukaryotic host will have a genetic code compatible with the expression of higher eukaryotic DNA sequences, however, as would be appreciated by one skilled in the art, a lower eukaryote with a non-standard genetic code, for example ciliates, may be used provided the vitellogenin gene sequence is appropriately modified to allow expression under this alternate genetic code.

The eukaryotic host according to various embodiments of the invention may be used as a direct feed or as an indirect feed that is used to enrich the nutrient quality of other organisms in the food chain.

In a specific embodiment, the transgenic eukaryotic cell is a yeast cell, for example S. cerevisiae or P. pastoris. P. pastoris is particularly preferred since, as indicated above, this host has (1) the ability to grow to high cell densities using inexpensive simple, defined media; (2) the capacity to express high levels of recombinant proteins when multiple copies of an expression cassette are chromosomally integrated; and (3) known to carrying out eukaryotic post-translational modifications (Clare et al, 1991a; 1991b; Cregg et al. 1993; Romanos 1995. In another specific embodiment, the transgenic yeast cell is protease deficient, for example P. pastoris strain SMD1168H.

A specific embodiment of the invention relates to a transgenic SMD1168H strain of Pichia pastoris expressing vitellogenin protein, wherein the expression of rVtg, is driven by a glyceraldehyde-3-phosphate dehydrogenase (GAP) promoter (Waterham et al., 1997). The GAP promoter offers an attractive alternative to the alcohol oxidase 1 (AOX1) promoter for the heterologous expression of some recombinant proteins. In the presence of glucose, expression under the control of GAP promoter is significantly higher than under the commonly used AOX1 promoter (Waterham et al., 1997). The constitutive GAP promoter allows a simplified fermentation regime and avoids the use of methanol, which is potentially toxic for subsequent applications. In one embodiment, the vitellogenin gene is from O. aureus.

The transgenic host according to the invention, including transgenic yeast described herein may be used as a feed alone, or co-fed with another food source. The specific transgenic host should be capable of being digested by the animal to which it is fed, and preferably is a natural feed source for the intended animal. For example, if the feed is intended for fish, the transgenic host may be algae or yeast, both of which have been used as feed in aquaculture. Alternatively, trangenic algae or yeast may be used to enrich another food source for fish such as rotifers or artemia (intermediate live feed as they are known in the art). High protein yeast feed has also been used for other domestic animals (Chen et al., 2000). In a specific embodiment, the methylotrophic yeast Pichia pastoris transformed according to the invention is used as a live feed or in dry form. In another specific embodiment, the Pichia pastoris strain is protease deficient, such as P. pastoris SMD1168H.

Nutritional analyses show that transgenic yeast clones according to the invention expressing rVtg contain increased levels (meaning a detectable increase when compared to a control native yeast strain) of essential amino acids and fatty acids, and these recombinant clones may therefore be advantageously used in aquaculture either as a direct probiotic feed, a feed additive for formulated diets or as primary feed for live intermediate feed hosts such as rotifers or artemia. In one aspect of the invention, the transgenic yeast and other hosts according to various embodiments of the invention are used to enrich an intermediate live feed such as Artemia nauplii and in one embodiment, the polyunsaturated lipid content of artemia is increased.

The level of important n-3 polyunsaturated fatty acids (PUFA) can be drastically elevated by culturing the yeast clones in the presence of fish oil. As PUFA's are important components of cellular and structural function, such transgenic yeast may be particularly advantageous as a feed or feed additive. Another aspect of the invention therefore relates to a method of increasing polyunsaturated fatty acid content in the transgenic eukaryotic host according to the invention comprising culturing the host in a media comprising fish oil. The fish oil may be pure cod liver oil containing approximately 8% (w/v) eicosapentaenoic acid and 7% (w/v) docosahexanoic acid, or other similar fish liver oils. In a specific embodiment, the culture media includes about 2-5% of fish oil.

In another aspect, the invention relates to a method for increasing the survival rates of oviparous larvae comprising the step of feeding the larvae with a transgenic yeast or other host or an intermediate live feed that has been fed with a transgenic yeast or other host according to various embodiments of the invention. In various embodiments the method relates to increasing the survival rates of the larvae of aquatic or marine oviparous animal, such as fish, for example tilapia. The amount of transgenic yeast administered in the method will depend on the nature of the larvae to be fed. For example, the transgenic yeast may be administered to tilapia larvae in an amount corresponding to about 1.0 to 1.6 mg of dried yeast per tilapia larvae per day. In another specific embodiment of the invention, the transgenic yeast may be co-fed with another food source such as rotifers or artemia. The amount of the other food source will depend on the nature of the oviparous larvae being fed. For example, where tilapia larvae are co-fed with rotifers, the rotifers may be fed to the tilapia larvae at a density of 5 individuals per milliliter of the volume of the vessel containing the tilapia larvae. For other larvae, a person skilled in the art would know the appropriate amount of the other food source.

In yet another aspect, the invention relates to a method of enriching an intermediate live feed, for example artemia or rotifers, comprising the step of feeding to the intermediate live feed a transgenic yeast or other host according to various embodiments of the invention. In a specific embodiment, the total lipid content of Artemia nauplii is increased by feeding about 0.5 μg of the transgenic yeast according to various embodiments of the invention per Artemia per day.

In another aspect, the invention relates to a method for increasing broodstock egg quality of an oviparous animal comprising the step of feeding the broodstock a transgenic yeast or other host or an intermediate live feed that has been fed a transgenic yeast or other host according to various embodiments of the invention. In a specific embodiment, the broodstock are aquatic or marine oviparous animal, for example fish, such as tilapia. The amount of transgenic yeast administered will depend on the nature of the broodstock being fed and whether the transgenic yeast of the invention are employed alone or co-fed with another food source.

In another aspect, the invention relates to use of recombinant vitellogenin for delivery of a therapeutic material to the maternal oocytes of an oviparous animal. A therapeutic material as used in this context describes any material that enhances the survival and growth of broodstock and includes hormones, vitamins, minerals, ions, and nucleic acid. For example, if the oocytes of a broodstock animal contains a suboptimal amount of any number of nutrients capable of being bound by vitellogenin, such as minerals, ions, or vitamins, these nutrients may be selectively delivered to oocytes by mixing such materials with purified vitellogenin and subsequently administering the resulting vitellogenin complex to the desired broodstock, for example by injection into the bloodstream of the maternal broodstock animal, where the vitellogenin complex is subsequently taken up by oocytes by Vtg receptor mediated-endocytosis.

As can be understood by one skilled in the art, many modifications to the exemplary embodiments described herein are possible. The invention is intended to encompass all such modifications within its scope, as defined by the claims.

The documents referred to herein are fully incorporated by reference.

### EXAMPLES

Bacto yeast extract, bacto-peptone, yeast nitrogen base, bacto-agar, and skimmed milk were obtained from Difco, USA. Zeocin was from Invitrogen, USA and zymolyase was from Seikagaku Corp., Japan. DIG-labelling kit was obtained from Roche, GmbH. Goat anti-rabbit HRP-conjugated antibody was a product of Dako, Denmark, and supersignal chemiluminescent substrate was purchased from Pierce, USA. Protein A agarose and ³H-leucine were from Zymed (USA) and Amersham (UK), respectively. Fish oil, containing pure cod liver oil with 8.28% (w/v) eicosapentaenoic acid (EPA) and 7.36% (w/v) docosahexaenoic acid (DHA) was from Seven Seas, UK.

### Example 1

### Construction of the Expression Vectors & Transformation of Pichia pastoris

P. pastoris vectors, pGAPZA and pGAPZαC, were from Invitrogen (USA). The plasmids harbour a dominant selectable shuttle marker, Zeocin, which allows selection of both E. coli and P. pastoris transformants. While pGAPZA contains no secretion signal, pGAPZαC carries S. cerevisiae α-factor secretion signal sequence downstream of GAP promoter. The constitutive GAP promoter allows a simplified fermentation regime over the methanol-inducible AOX1 promoter by avoiding the use of methanol, which is flammable and is potentially toxic for subsequent applications.

The tilapia, O. aureus, Vtg cDNA (GenBank pOAVtg1 sequence AF017250) was inserted in the sense orientation, downstream of the GAP promoter of pGAPZA and pGAPZαC to create three constitutive Vtg expression vectors. Prior to subcloning into the GAP vectors, the native TAA stop codon of the Vtg gene was altered to introduce an ApaI restriction site to enable subsequent in-frame fusion with the myc-His C-terminal tags in the GAP vectors. PCR-based mutations of the cDNA were manipulated in pBlueScript (pBSIISK) vector using the QuikChange™ Site-Directed Mutagenesis kit (Stratagene, USA). The two primers used were the T7 forward primer and 5′-C CAT GGG CCC AGC ACA CTG AGG AGT GCA GC-3′, where the ApaI site is underlined (SEQ ID NO: 21). The PCR product was sequenced to confirm the change of TAA and TCT codons to GGG and CCC, respectively.

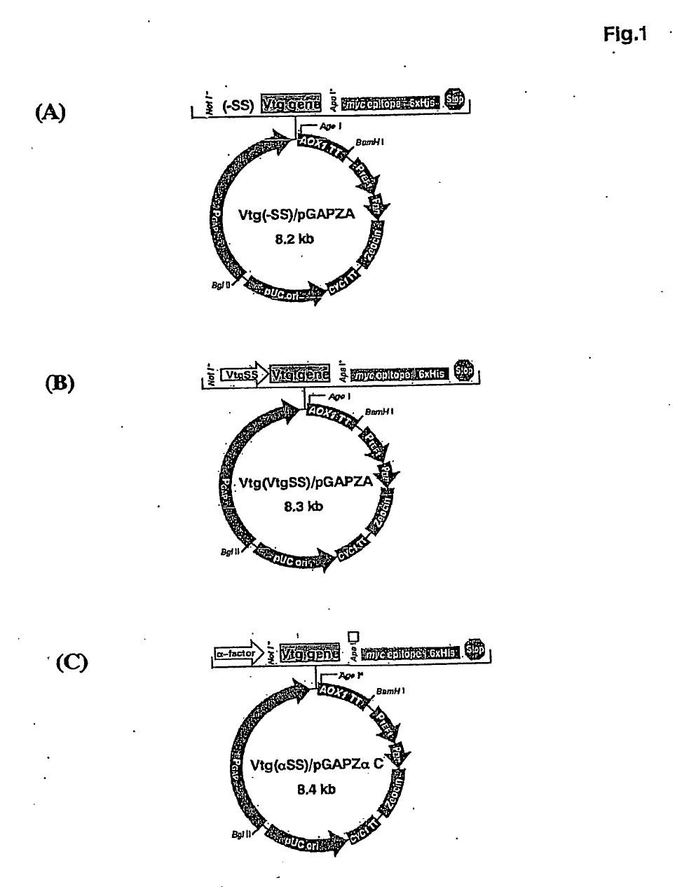

Vtg(-SS)/pGAPZA is the plasmid for constitutive intracellular expression. Prior to insertion into pGAPZA, the 5′ region of the cDNA where the native VtgSS ends was mutated to create an SpeI site preceding a new internal start codon. Primers used were 5′-T CTT GCT GTG GCT CTA CTA GTG ATG GAC CAG TCC AAC TTG GCC-3′ (SEQ ID NO: 22) and 5′-C CAA GTT GGA CTG GTC CAT CAC TAG TAG AGC CAC AGC AAG AGC-3′ (SEQ ID NO: 23). The SpeI restriction sites are underlined and the new ATG codons are bold. In the SEQ ID NO: 22 primer, the codons for the 12^{th} and 13^{th} residue from the translation start site were both changed, from CTC (Leu) and GCA (Ala) to CTA, to create the SpeI site. An intentional codon change of the last residue of the VtgSS from GGG (gly) to ATG (met) was made to create a new internal start codon. By digesting the mutated pOAVtg1 with SpeI, the fragment flanked by two SpeI sites, one from the mutated SpeI and the other from the multicloning region of pBSIISK, was lost together with the VtgSS, and the plasmid was religated. Finally, the pOAVtg1 with mutations at both 5′ and 3′ ends was digested with NotI and ApaI and the resulting 53 kb fragment was inserted between the NotI and ApaI sites of pGAPZA to create Vtg(-SS)/pGAPZA. A schematic representation of the Vtg(-SS)/pGAPZA plasmid is depicted in FIG. 1A.

Vtg(VtgSS)/pGAPZA was intended for constitutive expression and secretion of rVtg using native VtgSS. In this vector, a OaVtg cDNA of 5.4 kb with an intact 5′ VtgSS but a mutated 3′ TAA codon was inserted between the NotI and ApaI sites downstream of the GAP promoter in pGAPZA. A schematic representation of the Vtg(VtgSS)/pGAPZA plasmid is depicted in FIG. 1B.

Vtg(αSS)/pGAPZαC was intended for constitutive expression and secretion of rVtg using the S. cerevisiae α-factor secretion signal (αSS). To construct this, the NotI-AgeI DNA fragment from (A) was inserted in the similar way into the pGAPZαC vector. A schematic representation of the Vtg(αSS)/pGAPZαC plasmid is depicted in FIG. 1C.

All plasmids were prepared by alkaline lysis method and further purified through a CsCl/ethidium bromide gradient-ultracentrifugation. To direct integration of the Vtg expression cassettes, BlnI-linearised plasmids (10 μg each) were added to 80 μl of competent P. pastoris (Higgins and Cregg, 1998). Transformation was effected by electroporation in a 0.2 cm electroporation cuvette, using the BioRad Gene Pulser at 1500 V, 25 μF and 200Ω. Transformed colonies were selected on YPD agar plates (1% yeast extract, 2% each of peptone, dextrose, 1 M sorbitol and 2% agar) containing 100, 500 or 1000 μg/ml Zeocin and incubated at 30° C. for 4 days.

Preliminary results showed that none of the clones harbouring secretion signal (VtgSS or αSS) secreted rVtg. Regardless of the SS, the rVtg remained within the yeast, in cell lysate and membrane-bound fractions. Nevertheless, this may be advantageous for its application as a recombinant feed. Since Vtg(αSS)/pGAPZαC constructs yielded only minimal number of clones with poor viability, further experiments were carried out with the Vtg(-SS)/pGAPZA and Vtg(VtgSS)/pGAPZA clones.

### Example 2

### Southern and Western Analyses of Vtg Gene Copy Number and Expression Levels

It has been reported that the expression levels of a recombinant protein in Pichia can be enhanced dramatically with multicopy transformants (Vassileva et al., 2001). Thus, we investigated the effect of gene copy number on the expression efficiency of the GAP-regulated rVtg constructs. The putative recombinant clones harboring multiple copies of the expression cassette were isolated by selection with increasing concentrations of Zeocin.

Yeast transformants were spheroplasted using zymolyase, and lysed using 1% sodium dodecyl sulfate (SDS). Genomic DNA was isolated by ethanol precipitation and resuspended in TE buffer, pH 7.5. The AvrII-digested genomic DNA samples (10 μg each) were electrophoresed on a 0.6% agarose gel and transferred onto 0.45 μm nylon membrane (Pall Biodyne, USA). The Southern blot was hybridised with a DIG-labelled O. aureus Vtg XhoI-XbaI fragment of 926 bp, excised from pOaVtg1 (Lim et al., 2001). For normalization, the blot was simultaneously probed with a histidinol dehydrogenase 4 (HIS4) gene fragment (612 bp) from the pPIC9 vector (Invitrogen), to detect the presence of the single-copy chromosomal HIS4 gene. To determine the copy number of the OaVtg cDNA integrated in the yeast host genome, the ratio of the intensity of the Vtg specific band and that of the HIS4 specific band was obtained by densitometric scan using Image-Master VDS software (Pharmacia Biotech). For normalization of the ratio of intensity, DNA from the untransformed host was also included in the analysis.

The total protein in culture supernatant, cell lysate and membrane bound fractions were quantified by Bradford assay (Bradford, 1976). The rVtg protein in each fraction was analyzed by SDS-PAGE and immunoblotting (Burnette, 1981) with some modifications. Fixed amount of 35 μg of total protein from the lysate and membrane fractions prepared from shake flask cultures of rVtg clones was loaded on a 10% SDS-polyacrylamide gel. The proteins were transferred by electroblotting. To detect rVtg, rabbit anti-Vtg antibody (1:10000) was used as primary antibody, with goat-anti-rabbit-HRP (1:20000) as secondary antibody, in the presence of 5% skimmed milk. The chemiluminescent substrate, Supersignal West Pico was used to develop the signal.

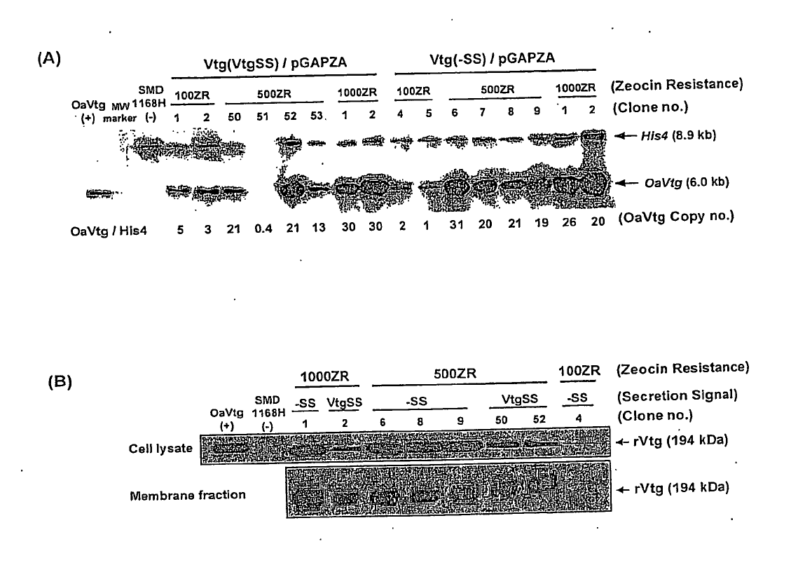

Southern blots of genomic DNA to determine the copy number of Vtg expression cassettes in Vtg(VtgSS) and Vtg(-SS) clones are shown in FIG. 2A. P. pastoris genomic DNA isolated from clones that survived selection with increasing zeocin concentrations (100, 500, and 1000 μg/ml) was digested and probed as described above. The numbers below the blot indicate the copy number of the Vtg expression cassettes based on the ratio of the densitometric intensity of the Vtg bands to those of the His4.

There is an apparent correlation between the increasing Zeocin tolerance (from 100 to 500 to 1000 μg/ml) and the copy number of Vtg expression cassettes (FIG. 2A). Thus, the clones with high copy numbers were named according to their levels of Zeocin Resistance (ZR): (a) clones harbouring Vtg(-SS)/pGAPZA were #1, #2 (1000ZR); #6, #7, #8, #9 (500ZR). (b) clones harbouring Vtg(VtgSS)/pGAPZA were #1, #2 (1000ZR), #50, #52 (500ZR).

Western analyses of the cell lysate and membrane fractions from the selected ZR clones, detected an rVtg band of 194 kDa (FIG. 2B). The combined levels of rVtg in these two fractions give the total intracellularly expressed rVtg. Amongst these clones, the highest integrants, represented by clones #6 and #52, harbored up to 31 and 21 copies respectively, of Vtg expression cassettes, and they expressed the highest combined (lysate and membrane-bound) levels of rVtg (FIG. 2B). The intensity of the bands compares closely with the copy number of the expression cassettes in the corresponding clone. Henceforth, we focused on investigating clones #6 and #52. Since clone #6 grows most robustly, biochemical analyses were performed on this clone.

### Example 3

### Effects of Media Composition on the Expression Level and Integrity of rVtg

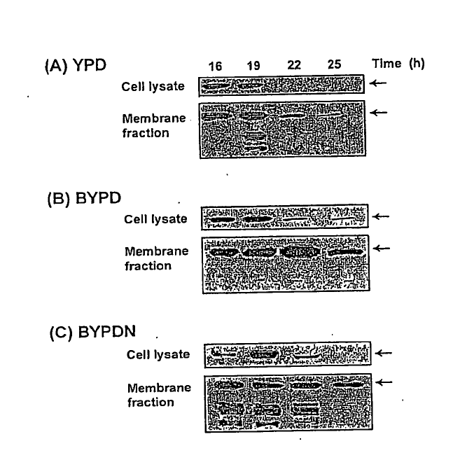

The expression levels of rVtg in cultures of clone #6 containing the Vtg(—SS)/pGAPZA plasmid were determined in three different YPD-based media: (a) YPD containing 1% yeast extract, 2% peptone and 2% dextrose, pH 6.0; (b) buffered YPD (BYPD) containing YPD supplemented with 100 mM phosphate buffer, pH 6.0; and (c) BYPDN containing BYPD, 1.34% yeast nitrogen base and 4×10⁻⁵% biotin, pH 6.0. Single colonies of the yeast were pre-cultured in 10 ml of each of the 3 media until OD₆₀₀ₙₘ of ˜7.0. One ml of this starter culture was inoculated into 200 ml fresh media in a 1 L shake flask for overnight incubation at 23° C., with continuous shaking at 260 rpm. The optimal time for the growth of clones was also monitored at 25° C., 28° C. and 30° C. From this culture, aliquots of 30 ml were harvested at 16, 19, 22 and 25 hr time points. The cells were pelleted at 5000 g for 5 min. The culture supernatant was set aside for analysis of secreted rVtg. The cell pellet was washed in breaking buffer (50 mM sodium phosphate, pH 7.4, 1 mM PMSF, 1 mM EDTA and 5% glycerol) and resuspended in 7.5 ml of breaking buffer. The cells were lysed either by glass beads (0.45 μm, Sigma) or French Press (Basic Z 0.75 KW Benchtop Cell Disruptor, UK) operated at 30 kpsi. After 1 h centrifugation at 14000 g at 4° C., the cell lysate and debris were separately stored at −80° C. for further analyses. The debris was solubilised with 1% SDS to obtain the membrane bound fraction. The Western blot analysis of the cell lysate and membrane fractions were as described above and the position of the 194 kDa vitellogenin band in each of the panels is indicated by the arrow (←). In all three media, it appears that the majority of rVtg is localised to the membrane fractions. The buffered media, BYPD and BYPDN, supported optimal growth of the recombinant yeast and conferred maximal yield of total cell protein. Considering the combined intensity of rVtg in the cell lysate and membrane-bound fractions, higher expression levels were sustained in BYPD (FIG. 3B) and BYPDN (FIG. 3C) than YPD (FIG. 3A). However, degraded bands of rVtg are more prominent in the YPD and BYPDN media. Since culture in BYPD maintained rVtg with highest integrity, further studies employed this medium.

### Example 4

### rVtg Expression in Single and Multiple Shake Flasks, and Fed-Batch Fermentation Culture of Clone #6

For the single shake flask experiments, Clone #6 was grown at 23° C. in a single 1 L baffled shake flask containing 200 ml of BYPD medium with shaking at 260 rpm. When tested over 23, 25, 28 and 30° C., the multicopy clones expressed optimally at 23° C. This is in agreement with the report of Li et al. (2001). At each time point, 30 ml cells were harvested with concurrent top-up of 30 ml of fresh BYPD after each sampling.

For the multiple shake flask experiments, seven replicates (one for each time point) of 1 L baffled shake flasks, each containing 200 ml of BYPD, were simultaneously inoculated with clone #6. The cultures were grown under the same conditions as the single shake flask culture. At each time point, 30 ml culture was sampled from one of the flasks.

For the fed-batch fermentation experiments, a single colony of yeast was inoculated into 10 ml BYPD medium and grown overnight at 23° C. in a shaker incubator at 260 rpm, to achieve OD₆₀₀˜7.0. The culture was inoculated into 100 ml of BYPD in a 1 L shake flask and grown to same density before inoculation into 1 L BYPD contained in a 2 L fermentor flask (BioSTAT, B. Braun). The dissolved oxygen level was maintained above 20% by air saturation at a rate of 2:1 (v/v) of air to fermentor per minute. The pO2 cascade was maintained at consistent level with appropriate stirring speed. At various time intervals, 30 ml of culture was sampled for analysis and an equal volume of fresh medium was introduced to maintain the original culture volume.

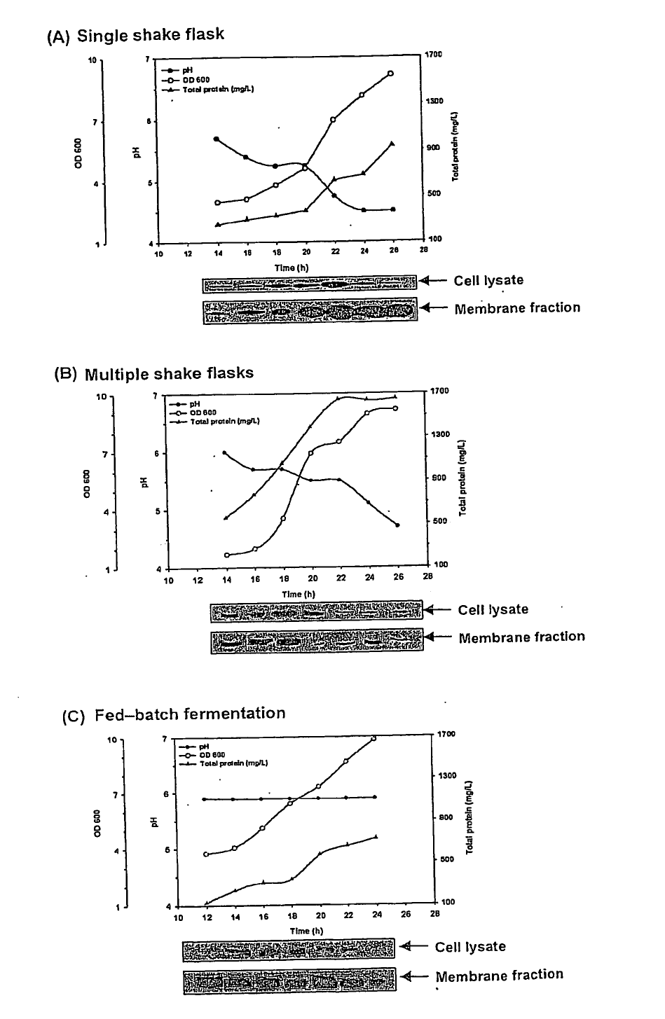

The rate and peak of expression of rVtg were monitored for each culture condition as shown in FIG. 4, where the pH (●), OD 600 nm (∘) and total protein (▴) of Clone #6 cultured under the different conditions are shown as a function of time. The Western blot presented below each growth curve in FIG. 4 shows that rVtg was detected in the cell lysate and membrane fractions. Comparison of the three Western blots revealed an apparent forward shift in the time of maximal rVtg protein accumulation from 22 h for the single flask, to 18 h for the multiple flasks and the fastest time of 16 h for the fed-batch fermentation cultures. Maintaining the pH near 6 appears to be important for prolonging exponential cell growth (refer to FIG. 4C).

As shown above, the recombinant yeast clones are amenable to continuous fermentor culture, permitting in principle, indefinite rVtg expression. Theoretically, the expression level can be maintained constant in the fermentation culture due to the constitutive property of the GAP promoter. However, rVtg level in fed-batch fermentation culture was maximum at 16 h, achieving peak concentration earlier than the shake flask cultures, but diminished thereafter. This is despite the maintenance in fermentor culture of constant pO₂ and pH. It is unlikely that the Vtg gene was lost after the peak of expression, since further inoculations of the fermentor clones into fresh BYPD again showed similar profiles of rVtg expression, thus, indicating that the integrated Vtg gene was still intact and expression competent. Furthermore, the clones were found to be stable and continuously and constitutively express rVtg over 2-3 years of culture since its existence. A plausible explanation may be depletion of the carbon source, and low saturation of the culture medium could have occurred after expression. It would be of interest in future, to monitor the levels of Vtg mRNA in the clones over these time intervals and beyond, to determine the point at which the Vtg expression was ceased, viz., the transcriptional or translational levels. This will help to address any potential concerns over the transcriptional and/or translational stress, where the Vtg mRNA stability and Vtg protein integrity, respectively, may become questionable. While this experiment indicates that variations in the culture conditions can drastically affect the expression level of rVtg in P. pastoris, a person skilled in the art can take necessary steps to ensure desired level of rVtg expression.

Immunoprecipitation of ³H-protein to determine the level of rVtg was performed as follows. Overnight cultures in BYPD were pelleted and washed twice with breaking buffer, resuspended in 50 ml BYPD containing 10 μCi ³H-leucine (specific activity 5 mCi/ml) and incubated with shaking at 260 rpm at 23° C. for 22 h. The cells pelleted from 15 ml of the culture were lysed using glass beads and centrifuged for 10 min at 9000 g. The supernatant contains the cell lysate. The pellet was solubilised with 0.1% SDS at 4° C. for 2 h and centrifuged. The resulting supernatant represented the solubilised membrane fraction.

Subsequently, 100 μL of cell lysate and membrane fraction were preincubated separately with 20 μl preimmune serum for 60 min at 4° C., following which 20 μl of protein A-agarose was added and allowed to react for 60 min at 4° C. The samples were centrifuged at 12000 g for 5 min. To the supernatant, 100 μl of anti-Vtg antibody (1:100) was added followed by overnight incubation at 4° C. After addition of 30 μl of protein A-agarose, the mixtures were centrifuged at 12000 g for 5 min. The immunoprecipitate was washed thrice with immunoprecipitation buffer containing 50 mM Tris, pH 7.5, 50 mM EDTA, 150 mM NaCl, and 1% NP-40. The resulting immunoprecipitate was treated with TCA (Melo et al., 2000). An aliquot of 50 μl of the radiolabelled cell lysate and membrane fractions were spotted on a 3MM Whatman filter paper and dried at room temperature. The filter papers were washed once with cold 10% TCA for 25 min and twice with 5% TCA followed by ethanol:ether (1:1) and finally with ether for 15 min. After drying, the radioactivity of ³H-labelled Vtg was counted in Aquasol using Wallac 1414 Liquid Scintillation counter (Beckman).

Radiolabelling of total protein in clone #6 showed 46.2% protein in the cell lysate and 53.8% in the membrane fraction. Immunoprecipitation of the radiolabelled rVtg by Vtg-specific antibodies showed that rVtg represents 1.16% of total protein (Table 1). The rVtg partitioned into the soluble cell lysate (0.44%) and membrane fractions (0.72%).

### Example 5

### Amino Acid Analyses of Clones #6 and #52

Unless otherwise stated, the growth and harvest of all P. pastoris cultures were performed according to the respective optimal conditions determined for the single shake flask, multiple shake flasks and fed-batch fermentation methodology (refer to FIG. 4). Shake flask cultures of clones #6 and #52 and the control SMD 1168H were harvested, washed and freeze-dried. One mg of freeze-dried yeast cells from the respective time points of different cultures was digested for 22 h with 6 N HCl at 110° C. under vacuum. HCl was evaporated from the sample by passing through nitrogen gas. The dried samples were resuspended in 2 ml of 0.2 M sodium citrate, pH 2.2 and filtered through a 0.22 μm filter. Amino acid analysis was carried out using Shimadzu Amino Acid Analyzer, LC-6A.

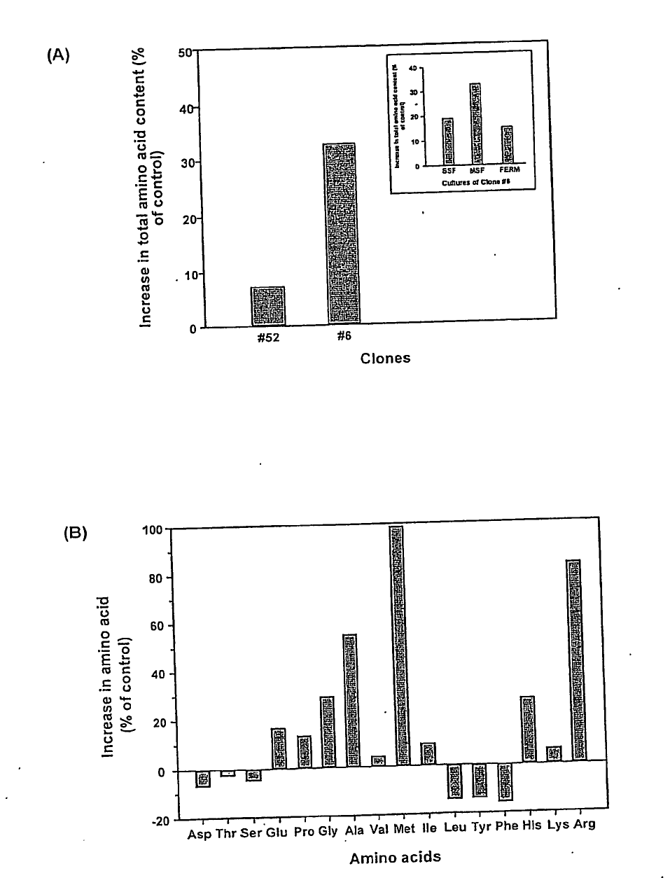

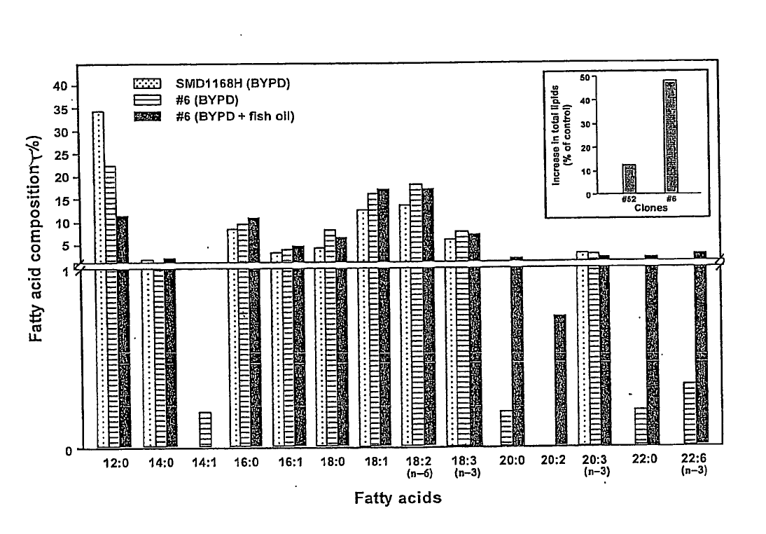

The amino acids values were expressed as nmol/mg dry yeast. All results were normalized against the native P. pastoris, SMD1168H. The percentage increase of total amino acid content in clones #6 and #52 is depicted in FIG. 5A. Compared to the host SMD1168H, clone #6 shows a 33% rise in total amino acid content, in contrast to the modest 6% increase in clone #52. The inset of FIG. 5A compares the profiles of percentage increases of total amino acid content amongst cultures of clone #6 in single shake flask (SSF; 19% increase), multiple shake flasks (MSF; 33% increase) and fed-batch fermentor (FERM; 15.3% increase). The percentage increase of individual amino acids in clones #6 cultured in multiple shake flasks is depicted in FIG. 5B.

Examination of the amino acid profiles showed that amongst the 16 amino acids analyzed, the content of 10 amino acids (glu, pro, gly, ala, lys, val, met, ile, his and arg) were higher in clone #6 compared to the control host SMD1168H (FIG. 5B). The latter 6 amino acids are generally considered as essential amino acids for most fish (Cowey and Cho, 1993, De Silva and Anderson, 1995). Amongst the individual amino acid analyzed in clone #6, arginine and methionine were most significantly enriched (FIG. 5B). Most noteworthy is the 98% increase in the methionine content. Considering that most formulated diets tend to be methionine-deficient, clone #6 appears to have great potential as a diet supplement.

### Example 6

### Fatty Acid Analysis of Clones #6 and #52

Lipids were extracted from the Pichia clones (Folch et al., 1957) using 2:1 v/v chloroform:methanol containing an anti-oxidant (made up with 1 g each of butylated hydroxyanisole and butylated hydroxytoluene in 100 ml chloroform:methanol mixture). Fatty acid methyl esters were prepared using boron trifluoride (AOAC, 1980). Separation of the methyl esters was carried out by gas-liquid chromatography on a Shimadzu GC-9A with a Thermon 3000A (Shimadzu) capillary column (2.5 m×0.25 mm i.d.). The temperature gradient was programmed at 4° C./min, over 160-220° C. and the flow rate of carrier gas, helium, was 0.63 ml/min. For the identification of fatty acids, a mixture of known fatty acids was used as standard (T. Watanabe, Tokyo University of Fisheries and Sigma).

In a separate experiment, clone #6 and the native host, SMD1168H, were grown in BYPD supplemented with sterile-filtered 2% cod liver oil, with a view to improving the fatty acid composition of the rVtg clone (Guo and Ota, 2000). The cells were washed thoroughly with 0.9% NaCl to remove any trace of adherent fish oil before lipid analysis.

Comparison of the total lipid content in clones #6 and #52 over native SMD1168H (inset) shows that clone #6 exhibited 47.5% boost in total lipid content, out-performing the 13% rise in clone #52. The bar chart in the main figure shows the comparison of the fatty acid composition between clone #6 and control SMD1168H, grown in BYPD with or without fish oil. The first and second numbers below the bar chart represent the numbers of carbon atoms and double bonds in the fatty acid chain and the pharmaceutically important omega 3 fatty acids (n-3) are denoted. Native SMD1168H contains much higher level of lauric acid (12:0) than clone #6. The levels of the myristic acid (C14:0), palmitic acid (16:0), palmitoleic acid (16:1) and the C18 PUFAs are comparable between clone #6 and the control. With the exception of C20:3 (n-6), the levels of C20 and C22 PUFAs are found in clone #6, but they are low or undetectable in the control. Interestingly, when clone #6 was supplemented with 2% fish oil as carbon source, the content of the C20 and C22 PUFAs was drastically enhanced. Therefore, the capability of clone #6 in incorporating PUFAs further promotes the application of the rVtg clone as a high quality broodstock diet supplement.

The level of important PUFAs could be drastically elevated by culturing the yeast clone in the presence of fish oil. Although information on the genome of SMD116811 is lacking, BLAST search using O. aureus Vtg gene against the S. cerevisiae genome identified a stretch of 100 amino acid residues within the O. aureus LVI that shows some similarity to the ABC family long-chain fatty acid transporter, Pxa1p, in S. cerevisiae. It is therefore tempting to speculate that this putative fatty acid binding site in the O. aureus LV1 that is integrated into the P. pastoris clone #6 is functional, thus contributing to the facilitated incorporation of long-chain PUFAs, especially docosahexaenoic acid (DHA, 22:6, n-3). These PUFAs have attracted considerable interest as pharmaceutical and nutraceutical compounds, and are important components of cellular structure and function. They are known to be required by rapidly growing fish larvae (Owen et al., 1975; Sargent et al., 1989; Watanabe, 1982). Owing to its ability to incorporate PUFAs, clone #6 is an excellent candidate for development into a high quality broodstock diet supplement. Therefore, careful considerations must be given to the culture condition, to attain the optimal expression rate, and nutrient value of this novel live recombinant yeast feed-package.

Although the specific amount of rVtg expressed was not high, the amino acid and lipid analyses showed significant increase in amino acids (33%) (see EXAMPLE 4) and lipids (47.5%), indicating the subtle nutrient potentials that clone #6 harbours as a direct single cell protein feed or as a primary feed for enriching rotifers to be used as a live feed for the developing fish larvae (De Silva and Anderson, 1995). The nutrient value is in agreement with other yeast products, which have been used as feed attractants (Sanderson and Jolly, 1994).

### Example 7

### Feeding Trials Using Clone #6 as Novel Nutrient Package

The rotifer, Brachionus plicalitis, was cultured in seawater (salinity 36 ppt) at 26-30° C. in 400 L fiber plastic tanks, for at least 3 months prior to testing. The rotifers were fed twice daily with 0.5 mg of commercial baker's yeast (DCL Yeast Ltd, UK) per million rotifers. The rotifers were filtered and inoculated to 20 L conical cylinder glass tanks to ensure no contamination over two weeks prior to feeding trial with clone #6.

Wild type Pichia pastons, SMD1168H (SMD) and recombinant yeast clone #6 were cultured as the negative control and test cultures, respectively, in BYPD as previously described. In separate cultures, 2% and 5% pure cod liver oil (SEVEN SEAS, UK)—containing BYPD medium was used to culture the clones for further nutrient enrichment. Cells were freeze-dried and the powder yeasts were sealed and kept at 4° C. All the cultures were tested by Western-blot to confirm rVtg expression.

Fertilized eggs from within the mouth of the tilapia fish (Oreochromis aureus) were taken periodically to allow artificial hatching in a tank, at a density up to 500 eggs/liter with gentle aeration. Dechlorinated water was used for the culture and the temperature was within 26-30° C. The water was renewed every day to avoid fungal contamination and to increase the hatching rate. Approximately 3 days after hatching, the larvae were counted to assess the hatching rate, and at the same time, they were randomly distributed into 3 L glass cylinder tanks containing 25 L water at a density of 10-20 individuals/L, with gentle constant aeration at 26-33° C.

The co-feeding experiments were performed as follows. 50 fish larvae were placed in the tank, and co-fed with rotifer (at a density of 5 individuals/ml) and yeast (either baker's yeast or SMD or #6). Either the commercial baker's yeast or the lab cultured yeasts, all in dried powders were fed at a ratio of 1 mg/larvae/day. The survival rate, length and weight of the fish were taken. Six experimental groups were set up:

(1) feeding with rotifer alone (control);

(2) feeding with 100% baker's yeast (control);

(3) co-feeding with rotifer and yeasts (75% baker's yeast+25% #6);

(4) co-feeding with rotifer and yeasts (50% baker's yeast+50% #6);

(5) feeding with the yeasts alone (25% baker's yeast+75% #6);

(6) co-feeding with 100% #6 alone (control).

The results of the co-feeding experiments after 15 days of feeding post hatching are reported in Table 2, where S.c. denotes Baker's yeast (S. cerevisiae); ind is an abbreviation for individual and #6 denotes transgenic P. pastoris clone #6.

TABLE 2

| | | | Total | | | |
|---|---|---|---|---|---|---|
| | Groups | Survival | Wet Wt. | Survival | Wet Wt. | Length |
| | (16 days) | No. | (mg) | Rate (%) | (mg/ind) | (mm) |
| 1 | Rotifer | 7/50 | 19.6 | 14 | 2.80 | 10 |
| 2 | Rotifer + 100% S.c | 32/50 | 148.5 | 64 | 4.64 | 11.1 |
| 3 | Rotifer + 75% S.c/25% #6 | 30/50 | 82.5 | 60 | 2.75 | 11.4 |
| 4 | Rotifer + 50% S.c/50% #6 | 38/50 | 137.5 | 76 | 3.62 | 10.75 |
| 5 | Rotifer + 25% S.c/75% #6 | 41/50 | 179.0 | 82 | 4.37 | 10.8 |
| 6 | Rotifer + 100% #6 | 36/50 | 176.6 | 72 | 4.90 | 10.8 |

The direct yeast feeding experiments were performed as follows. 38 fish larvae were inoculated into the 2.5 L water in the 3 L tanks. Yeasts were offered at the ratio of 1.6 mg yeast/larvae/day. Three groups were set up:

(1) 100% baker's yeast;

(2) 100% SMD1168H

(3) 100% of #6.

Results of the direct feeding experiment after 48 days of feeding are reported in Table 3.

TABLE 3

| | | | | | Mean |
|---|---|---|---|---|---|
| Group | Survival | Survival | Wet Wt | Wet Wt | Length |
| (49 days) | No. | rate (%) | (mg) | (mg/ind) | (cm) |
| S.c | 4/38 | 10.53 | 139.20 | 34.80 | 1.43 |
| SMD | 8/38 | 21.05 | 220.20 | 27.53 | 1.33 |
| #6 | 17/38 | 44.74 | 568.40 | 33.44 | 1.42 |

The oil-enriched yeast feeding experiments were performed as follows. 32 fish larvae were randomly distributed into 2 of the 3 L tanks with 2.5 L water. Two groups were set up and fed at ratio of 1.6 mg yeast/larvae/day:

(1) 100% of #6 cultured with 2% oil in BYPD

(2) 100% of #6 cultured with 5% oil in BYPD

Results for the oil-enriched yeast feeding after a 35 day feeding are reported in Table 4.

TABLE 4

| | | | | | Mean |
|---|---|---|---|---|---|
| | Survival | Survival | Wet wt | Wet wt | Length |
| Group | No. | rate (%) | (mg) | (mg/ind) | (mm) |
| #6 (2% oil) | 13/32 | 40.63 | 164 | 12.62 | 10.89 |
| #6 (5% oil) | 24/32 | 75.00 | 242 | 10.08 | 10.55 |

### Example 8

### Live Intermediate Feed Hosts

Materials

Artemia (Artemia salina) cyst was obtained from Biomarine, USA.

Pichia pastoris, SMD1168H (SMD), is the wild type yeast used as the negative control. FL(-ss)pGAPzA in SMD1168H/500ZR #6 is the optimal yeast Vtg expression (intracellular) clone tested. The yeasts were cultured as described previously. Recombinant yeast clone #6 was cultured in BYPD medium containing 5% pure cod liver oil. This culture is henceforth referred to as #6 5% fish oil. With clone #6 grown in fish oil, repeated washings were needed to remove the unconsumed oil. The cells were freeze-dried using a FLEX1-DRY™ (FTS, USA) freeze drier and sealed and kept at 4° C. For comparison, commercially available baker's yeast (Sacchacromyces cerevisiae, DCL, UK) in lyophilized form was used.

Summary: The potential of using recombinant vitellogenin in the yeast Pichia pastoris as a direct probiotic feed, or for Artemia enrichment as nutrient feed to fish larvae has been evaluated. The amount of total lipids of Artemia nauplii enriched with recombinant yeast vitellogenin clone #6 (or clone #6 cultured in 5% cod liver oil) increased by 2-fold from 34.23 to 68.27 mg/g compared to the wild type Artemia nauplii. Feeding Artemia with either clone #6 or clone #6 in 5% oil increased the level of highly unsaturated fatty acids especially eicosapentaenote (20:5 n-3; EPA) and docosahexaenoate (22:6 n-3; DHA). The level of EPA increased from 3.46±0.46 to 5.57±0.39% when compared to Artemia nauplii fed with wild type Pichia pastoris and Saccharomyces cervisiae. The concentration of DHA in Artemia nauplii enriched with clone #6 or clone #6 in 5% oil was 0.28±0.14 and 1.02±0.28, respectively, but the DHA was undetectable in the Artemia nauplii fed with P. pastoris and S. cerevisiae. The maximum length (10.8±0.7 mm) and weight (16.3±2.5 mg) were achieved with tilapia larvae fed with clone #6 or #6 5% oil. The percentage survival (85.3±1.5%) of the tilapia larvae was significantly (P<0.05) higher when fed with clone #6 or #6 in 5% oil compared to larvae fed with either S. cerevisiae or P. pastoris, although larval growth (length and weight) did not differ significantly. Co-feeding with wild type Artemia nauplii resulted in improvement of the larval growth and survival rate. Larvae fed 10 days with S. cerevisiae and another 10 days with enriched Artemia showed no significant difference in growth and survival. However, larvae fed 10 days with clone #6 and another 10 days with clone #6 or clone #6 in 5% oil-enriched Artemia achieved maximum length (18.2±05 mm), weight (89.6±8.3 mg) and survival (89.0±2.3). Thus, clone #6 and clone #6 in 5% cod liver oil could be used either directly as a high quality live-nutrient feed or indirectly via enrichment through Artemia nauplii, which is subsequently fed to the larvae, resulting in significant improvements in growth and survival.

Intermediate Live Food Culture—Artemia

Artemia cysts (Artemia salina) were hatched in a 15 L conical glass container filled with 10 L of filtered seawater. Strong aeration was provided and the cysts were incubated at room temperature (26-30° C.) for 36 h. Artemia nauplii were separated from the empty cysts and debris, and were transferred to clean filtered seawater with aeration. The Artemia at second instar stage were sampled and counted for estimation of density.

Enrichment of Artemia Nauplii with Yeasts (Wild Type and rVtg Clones)

The Artemia was enriched by feeding for 48 h with different yeast preparations: (1) baker's yeast; (2) SMD1168H; (3) clone #6 and (4) clone #6 in 5% fish oil. Each experiment was conducted in triplicate. The yeast was offered at a rate of 0.5 μg per Artemia per day. All freeze-dried yeasts were first mixed to homogeneity in a small amount of water. A quantity sufficient for 3-5 day feeding was prepared each time and was kept at −30° C. Samples of wild type and enriched Artemia nauplii were obtained for lipid analysis by collecting 50,000 Artemia per triplicate feed on a nylon mesh. The Artemia were rinsed using distilled water, blotted on absorbent tissue, transferred to a cryovial, and stored in −30

Tilapia Fish Larviculture

Tilapia Oreochromis mosambicus broodstock were collected from the culture tanks. Fertilized eggs within the mother's mouth were taken and were pooled to obtain the required quantity. They were then placed in a 3 L glass cylinder tank with gentle aeration. The water was changed every day to avoid fungal contamination and to increase the hatching rate. Hatched larvae were transferred to 3 L flat-bottomed glass cylinder tanks at a density of 30 larvae/L with gentle and constant aeration. Larvae were reared at 26-30° C. with continuous light.

Feeding Trials of Tilapia Larvae with Yeasts with/without Artemia Nauplii

Five different feeding experiments were carried out to evaluate the effects of the feed on growth and survival rates of Oreochromis mosambicus larvae. All experiments used 3-day posthatched fish larvae at a density of 30/L. The first experiment involved feeding with four different yeasts: (i) S. cerevisiae; (ii) SMD1168H; (iii) clone #6 and (iv) clone #6 in 5% oil. In the second experiment, the larvae were fed with three different combinations of yeasts: (i) 50% S. cerevisiae+50% SMD1168H; (ii) 50% S. cerevisiae+50% clone #6; and (iii) 50% S. cerevisiae+50% of clone #6 in 5% oil. The third experiment involved feeding for the first 10 days with (i) S. cerevisiae; or (ii) SMD1168H; or (iii) clone #6 or (iv) clone #6 in 5% oil, followed by co-feeding with wild type Artemia nauplii (5 individuals/ml) for another 10 days. The fourth experiment involved feeding the larvae for 10 days with three different combinations of yeasts: (i) 50% S. cerevisiae+50% SMD1168H; (ii) 50% S. cerevisiae+50% of clone #6; (iii) 50% S. cerevisiae+50% clone #6 in 5% oil. After 10 days, the larvae were co-fed for another 10 days with wild type Artemia nauplii (5 individuals/ml). The fifth feeding regime was for first 10 days with: (i) S. cerevisiae; (ii) clone #6, followed by another 10 days co-feeding with Artemia nauplii (5 individuals/ml) which had been enriched previously with either S. cerevisiae, SMD1168H, clone #6 or clone #6 in 5% oil.

Growth and Survival Assessments of the Tilapia Larvae

At the end of the feeding regimes, the larvae were sampled and anesthetized by placing in beakers that were kept in ice. The standard length was defined as the length in millimeters from the tip of the head to the end of the tail. After morphometric measurements, the larvae were placed on paper towels to dry, and weighed. The survival was determined by counting the larvae after the end of feeding experiment.

Lipid and Fatty Acid Analyses

Total lipids from Artemia fed with wild type yeasts and Artemia enriched with Vtg yeast recombinant clones were extracted in chloroform/methanol according to Floch et al. (1957). Briefly, lipids were extracted from the Artemia using 2:1 (v/v) chloroform/methanol containing an antioxidant (made up with 1 g each of butylated hydroxyanisole and butylated hydroxytolune in 100 ml chloroform:methanol mixture). The fatty acids were converted to methyl esters by the Boron trifluoride (13%) method. The methyl esters of fatty acids were further separated by Gas-Liquid Chromatography on a Shimadzu GC-9A with a Thermon 3000A capillary column (2.5 m×0.25 mm i.d.). The temperature gradient was programmed at 4° C./min over 160-220° C. and the flow rate of the carrier gas helium, was 0.63 mL/min. For the identification of fatty acids, a mixture of known fatty acids was used as strandard (T. Watanabe, Tokyo University of Fisheries and Sigma Co).

Statistical Analysis

Diet-related differences in growth and survival profiles between treatments were analyzed using one-way ANOVA. Differences between means were compared using Newman-Keuls multiple comparisons test. A significance level of 95% (P<0.05) was used throughout.

Results

Artemia Nauplii Fed with rVtg Yeast Clone #6 Exhibits Increased Total Lipids

The total lipids in Artemia nauplii that has been starved for 48 b decreased by 38% from 34.23 to 21.15 mg/g (see Table 5). This indicates that, Artemia enrichment with any of the yeasts should always be applied since lipid contents in Artemia gradually decreases after hatching (Watanabe et al. 1982). The amount of total lipids of Artemia nauplii enriched with clone #6 or clone #6 in 5% fish oil increased from 34.23 to 68.27 mg/g compared to that of wild type Artemia.

The Fatty Acid Composition of the Cultured Artemia Nauplii

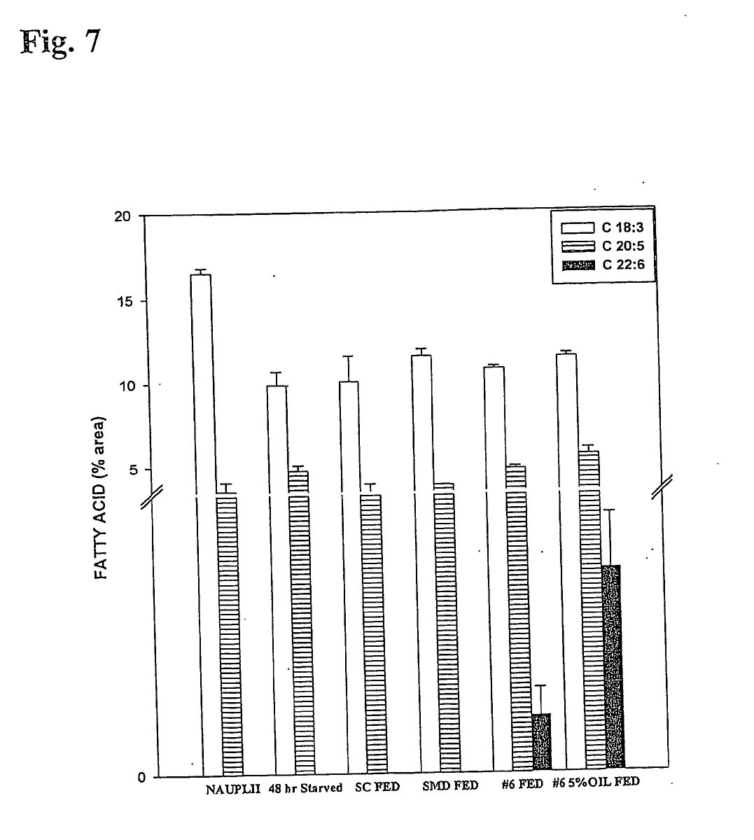

The fatty acid composition of Artemia nauplii (wild type) and Artemia nauplii fed for 48 h with baker's yeast (S. cerevisiae, SC), P. pastoris (wild type SMD1168H), rVtg clone #6 or clone #6 in 5% oil, are presented in Table 6. The amount of fatty acid 18:3 in Artemia starved for 48 h decreased from 16.5±0.3% to 9.9±0.8%. In contrast, the amount of fatty acid 20:5 increased from 3.5±0.6% to 4.7±0.3% (FIG. 7). Fresh Artemia nauplii contained 3.53±0.55% EPA but lacked DHA. Similarly, Artemia fed with SC & SMD1168H contained 3.46±0.46 and 3.85±0.02% EPA, respectively but lacked DHA. Interestingly, the levels of 20:5 (EPA) and 22:6 (DHA) were found to be higher in Artemia fed with clone #6 and clone #6 in 5% oil, but they were low or undetectable in the Artemia fed with control wild type yeasts (FIG. 7). The concentration of eicosapentaenoic acid (20.5 n-3) and docosahexaenoate (22.6 n-3) in artemia nauplii enriched with clone #6 and #6 in 5% oil were 4.77±0.14, 5.57±0.39 and 0.28±0.14, 1.02±0.28, respectively.

Larvae Fed with Clone #6 and #6 in 5% Oil Exhibited Higher Growth and Survival.

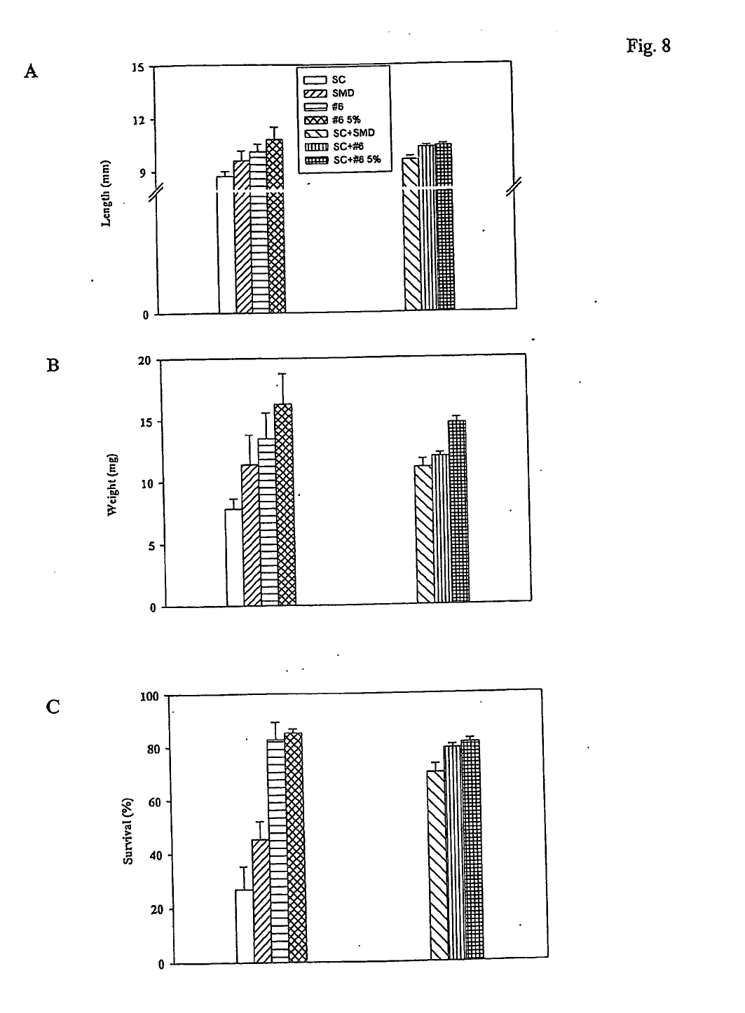

The maximum length, weight and survival were obtained with larvae fed with clone #6 or clone #6 in 5% oil for 20 days are shown in FIGS. 8 A & B. The survival of fish larvae fed with SC, SMD, #6 or #6 in 5% oil were 26.6±8.4, 45.3±6.7, 82.6±6.7 and 85.3±1.5%, respectively. The means survival for #6 and #6 in 5% oil fed larvae were statistically significant (p<0.05) than SC and SMD fed larvae. But there was no significant differences between SC and SMD fed larvae. The length and weight of fish larvae fed with SC, SMD, #6 or #6 in 5% oil were not statistically different from each other. On the other hand, supplementing with 50% S. cerevisiae results a 15% decrease in larval growth (length and weight). However, the survival rate increased by 5% (FIG. 8 C).

Larvae Co-Feeding with Clone #6 and Wild Type or #6 Enriched Artemia Exhibited Higher Growth and Survival.

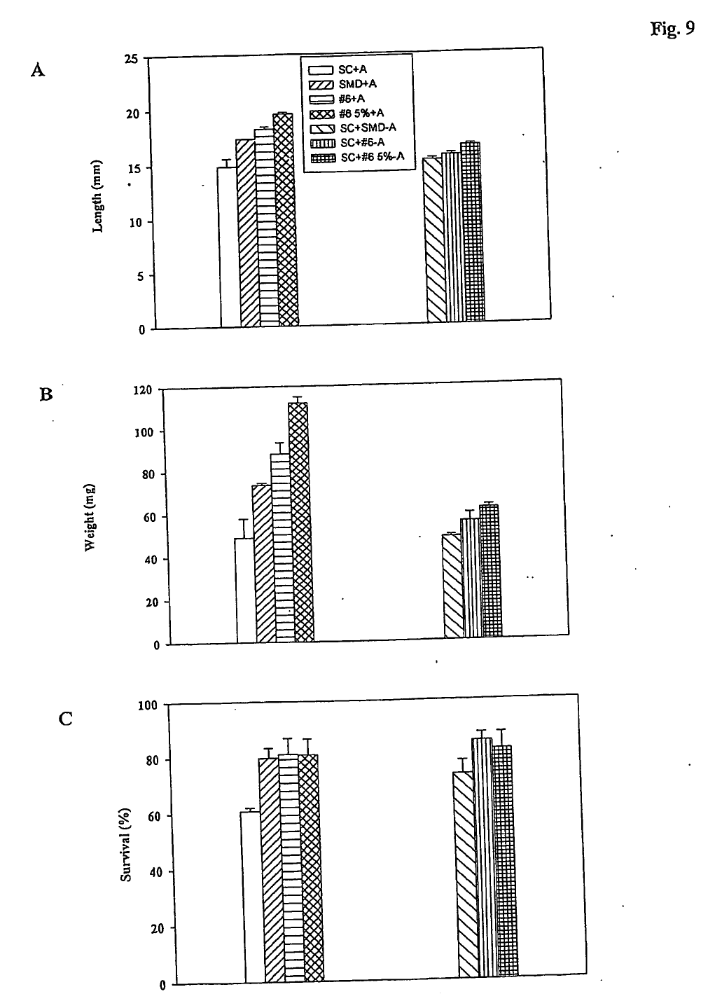

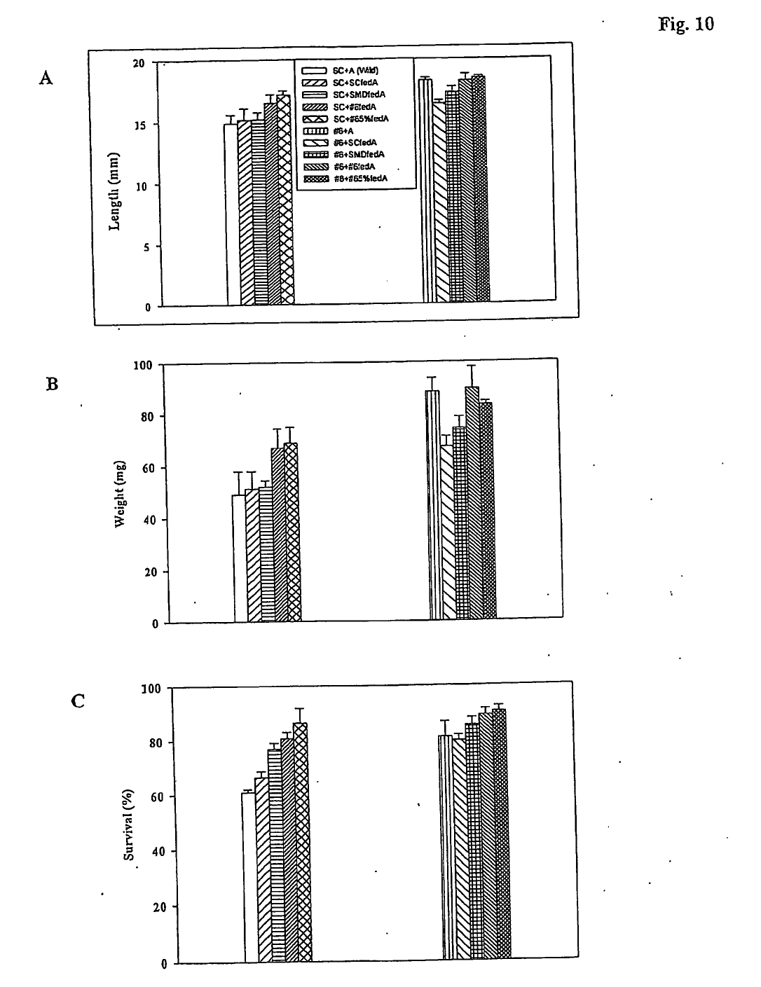

The co-feeding experiment clearly shows that the larvae fed for 10 days with clone #6 or #6 in 5% oil and subsequently for 10 days with Artemia (wild type) showed maximum length (19.6±0.1 and weight (112.2±2.9) when compared to other feeding regimes (FIGS. 9 A & B). The survival was also 21% (p<0.05) higher when compared between the larvae were fed 10 days with SC and clone #6 or #6 in 5% oil and subsequently 10 days with wild type Artemia (FIG. 9 C). Co-feeding with wild type Artemia resulted in improvement of the larval survival rate with SC and SMD yeast fed fish larvae. If we compare larvae fed 10 days with wild type yeast S. cerevisiae followed by another 10 days with enriched Artemia, there was no significant difference in length and weight between Artemia enriched with SC or with clone #6 (FIGS. 10 A & B). However, larvae fed for 10 days with clone #6 and another 10 days with enriched Artemia achieved maximum length (18.2±0.5) and weight (89.6±8.3) with Artemia enriched with #6 (FIGS. 10 A & B). Interestingly the highest survival was obtained with larvae fed for 10 days with clone #6 and another 10 days with Artemia enriched with clone #6 or #6 in 5% oil (FIG. 10 C).

TABLE 5
Total amount of lipids, % of total lipids and % increase in
total lipids in fresh, 48 hr starved and 48 hr fed with Baker's yeast SC
(Saccromyces crevisiae) SMD (Pichia pastoris), recombinant yeast
clone #6 or recombinant yeast clone #6 cultured in 5% cod liver oil
(#6 5% oil). Data are mean ± SE of triplicate sample.

| | Total lipids | Total lipids | % increase in |
|---|---|---|---|
| | (mg/g) | (%) | total lipids |
| Freshly hatched | 34.23 ± 1.14 | 3.42 ± 0.11 | — |
| Artemia nauplii | | | |
| 48 hr Starved | 21.00 ± 1.28 | 2.10 ± 0.12 | −38.6 ± 1.6 |
| Artemia nauplii | | | |
| SC fed | 48.76 ± 2.29 | 4.88 ± 0.23 | 43.7 ± 11.5 |
| Artemia nauplii | | | |
| SMD fed | 53.10 ± 3.49 | 5.31 ± 0.35 | 55.8 ± 15.6 |
| Artemia nauplii | | | |
| #6 fed | 65.03 ± 2.30 | 6.50 ± 0.29 | 90.3 ± 10.6 |
| Artemia naupli | | | |
| #6 in 5% | 68.27 ± 2.29 | 6.82 ± 0.23 | 99.5 ± 5.6 |
| fish oil-fed | | | |
| Artemia nauplii | | | |

TABLE 6
Comparison of major fatty acid (% mean ± SE) of newly hatched Artemia nauplii and Artemia
nauplii enriched by feeding 48 hr with baker's yeast SC (Saccromyces crevisiae), SMD
(Pichia pastoris), recombinant yeast clone #6 (#6) or recombinant yeast clone #6 cultured in
5% cod liver oil (#6 5% oil). Data are mean ± SEM of triplicate sample.

| Fatty | ARTEMIA | | | | |
|---|---|---|---|---|---|
| acid | (WILD) | SC | SMD | #6 | #6 5% Oil |
| C14:0 | 1.02 ± 0.40 | 0.48 ± 0.29 | 0.62 ± 0.10 | 0.60 ± 0.09 | 0.84 ± 0.28 |
| C16:0 | 9.68 ± 0.99 | 8.46 ± 0.22 | 9.32 ± 0.14 | 8.98 ± 0.41 | 8.89 ± 1.14 |
| C17:0 | 1.53 ± 0.05 | 2.14 ± 0.87 | 1.94 ± 0.39 | 1.97 ± 0.30 | 1.64 ± 0.21 |
| C18:0 | 3.91 ± 0.40 | 5.19 ± 0.73 | 5.44 ± 0.30 | 4.98 ± 0.52 | 5.37 ± 0.89 |
| ΣSFA | 19.34 ± 0.15 | 20.15 ± 0.13 | 20.41 ± 0.16 | 19.24 ± 0.07 | 21.74 ± 0.19 |
| C16:1 | 5.43 ± 0.20 | 14.59 ± 0.09 | 4.99 ± 0.31 | 4.35 ± 0.48 | 4.11 ± 0.19 |
| C18.1 | 36.08 ± 1.12 | 35.93 ± 2.94 | 34.27 ± 0.95 | 35.93 ± 0.01 | 35.40 ± 0.31 |
| C20:1 | 1.26 ± 0.15 | 1.15 ± 0.47 | 0.78 ± 0.20 | 0.73 ± 0.15 | 1.16 ± 0.47 |
| C22:1 | 0.83 ± 0.25 | 0.81 ± 0.33 | 0.53 ± 0.45 | 1.02 ± 0.29 | 0.87 ± 0.06 |
| ΣMUFA | 45.9 ± 0.23 | 57.35 ± 0.53 | 42.48 ± 0.17 | 42.96 ± 0.10 | 42.57 ± 0.08 |
| C18:2 | 3.57 ± 0.52 | 3.14 ± 0.89 | 4.90 ± 1.59 | 6.81 ± 1.59 | 6.72 ± 0.36 |
| C18:3 | 16.51 ± 0.28 | 10.02 ± 1.53 | 11.52 ± 0.41 | 10.72 ± 0.15 | 11.41 ± 0.20 |
| C18:4 | 2.42 ± 0.27 | 1.64 ± 0.40 | 1.53 ± 0.27 | 1.33 ± 0.40 | 1.64 ± 0.22 |
| C20:4 | 1.40 ± 0.34 | 2.19 ± 0.05 | 2.51 ± 0.40 | 2.62 ± 0.19 | 2.36 ± 0.14 |
| C20:5 | 3.53 ± 0.55 | 3.46 ± 0.46 | 3.85 ± 0.02 | 4.77 ± 0.14 | 5.57 ± 0.39 |
| C22:6 | | | | 0.28 ± 0.14 | 1.02 ± 0.28 |
| ΣPUFA | 29.39 ± 0.06 | 22.45 ± 0.25 | 27.0 ± 0.27 | 29.14 ± 0.23 | 30.41 ± 0.03 |

Discussion: One of the greatest obstacles to the mass production of very small, rapidly developing and highly vulnerable fish larvae is the high mortality that occurs in the first two weeks at the early stage of larval development during the transition from endogenous to exogenous food supply. A particular aspect of the problem is that the high growth and developmental rates of the larvae place a premium on providing optimal nutrition so that larval growth and development and, therefore, survival is maximum. Vitellogenin (Vtg) is a principal precursor of yolk proteins crucial for successful embryonic development and rapid growth of the larvae (Owen et al., 1975; Watanabe 1982; Sargent et al., 1989). Furthermore, the fish larvae require some form of live feed, like Artemia and rotifer during the earliest growth phases.

The small size and poorly developed digestive function of fish larvae (Segner et al., 1994) renders pelleted diets unsuitable for first feeding. The only viable alternative involves live-prey species such as Artemia nauplii or rotifers. Brine shrimp nauplii (Artemia spp.) are widely recognized to be an excellent starter feed for freshwater and marine fish species (Leger et al., 1986). Its nutritional value is strictly dependent on the nutrient quality of its food source, particularly the content of highly unsaturated fatty acids (HUFA), like docosahexaenoic acid (DHA, 22:6) and eicosapentaenoic acid (EPA, 20:5) (Scott and Middleton, 1979; Watanabe et al., 1983; Caric et al., 1993). It has been suggested that such HUFAs function as essential components of biomembranes (Koven et al., 1990), and their levels in the tissue phospholipid fraction are associated with larval growth. Sorgeloos et al. (1988) reported a strong correlation between the dietary EPA content and survival, and between DHA and growth of the Asian sea bass larvae. Watanabe (1993) concluded that DHA and EPA increased survival and growth of several marine fish larvae. However, these organisms are naturally low in long-chain polyunsaturated fatty acids (PUFA), especially DHA (Navarro et al., 1993; Copeman et al., 2002). Therefore, live feeds enriched with PUFA is highly desired. Lipids are particularly important in fish nutrition, not only for supplying caloric energy but also for providing the essential PUFAs required for normal cell membrane function (Sargent et al., 1995).

Several enrichment techniques have been developed, including microalgae, oil-based emulsions and microencapsulated preparations (Leger et al., 1986; Southgate and Lou 1995; Barclay and Zeller 1996; McEvou et al., 1996). Oil emulsions have been widely used for enrichment of live feeds in aquaculture (Dhert et al., 1990; Ostrowski and Divakaran 1990; Clawson and Lovell 1992; Stottrup and Attramadal 1992). Emulsion-type preparations have major advantages over dried enrichment diets as they have very high lipid and HUFA content. However, oil emulsions can be difficult to remove from Artemia following enrichment and this can result in transfer of oil, leading to water quality problems within larval rearing tanks (Rodgers and Barlow 1987; Rimmer and Reed 1990), and difficulties with swim bladder inflation in larval fish (Foscarini 1988).

Live nauplii of the brine shrimp (Artemia spp.) have been used as vectors for delivering compounds of diverse nutritional (Dhert et al., 1990; Tackaert et al., 1991) and/or therapeutic (Leger et al., 1987; Chair et al., 1991; Touraki et al., 1991; Cappellaro et al., 1993) values, which are otherwise difficult to administer to larval stages of aquatic animals, a process known as bioencapsulation. Bioencapsulated lactic acid bacteria have been successfully introduced into turbot larvae with significant improvements in survival (Garcia et al., 1992). This route has also been used to vaccinate sea bass fry (Chair et al., 1994), and juvenile carp (Joosten et al., 1995).

Before exogenous feeding, oviparous species of fish larvae are entirely dependent on their yolk supply for nourishment. Presumably, the lipids contained therein represent the optimum balance of fatty acids and other nutrients for the early stages of larval growth. The present study was designed to evaluate the nutritional value of the yeast recombinant Vtg (rVtg) as a direct probiotic feed, or for Artemia-enrichment as nutrient feed. We present data to demonstrate that the tilapia larvae fed with this novel yeast rVtg-enriched Artemia nauplii exhibit increased growth and survival.

Previous examples showed that the recombinant O. aureus Vtg yeast clone #6 exhibits significant increase in essential amino acids and long chain polyunsaturated fatty acids, for example, 20:5 n-3 and 22:6 n-3 (Ding et al., 2003). This study has shown that the engineered P. pastoris strain (#6) used as a direct feed or for HUFA enrichment of Artemia nauplii is an effective nutrient feed. The Artemia nauplii enriched with clone #6 supports greater growth of the tilapia larvae than wild type Artemia nauplii, which has significantly lower HUFA content.

The fatty acid composition of Artemia is probably the most studied biochemical component of this organism (Bengtson et al., 1991). In this study, the six fatty acids, 16:0; 16:1; 18:1; 18:2; 18:3; and 20:5 make up about 80% of the total fatty acids in all Artemia samples. A large database of published fatty acid profiles reveals that the above six fatty acids, normally make up about 80% of the total fatty acids in an Artemia sample (Leger et al., 1986). The saturated and monoene fatty acids of that group, 16:0, 16:1, and 18:1, typically comprise 40 to 60% of the total fatty acids. However, the n-3 series of fatty acids, linolenic acid 18:3 (n-3) is essential for fresh water organisms and 20:5 (n-3), is essential for marine organisms. The content of 20:5 (n-3) and 22:6 (n-3) of freshly hatched Artemia nauplii is low or even absent.

Artemia feeds on any particulate diet when its particle size is below 50 μm (Dobbeleir et al., 1980). Taking advantage of its continuous nonselective feeding behavior, Artemia nauplii fed for 48 h with #6 and #6 5% fish oil showed increase in the fatty acid content of 20:5 (n-3) and 22:6 (n-3). Pre-feeding with SC or SMD1168H lacking 20:5 (n-3) and 22:6 (n-3) did not increase the level of either of these fatty acids in the Artemia. When tilapia larvae were fed with #6-enriched Artemia or #6 in 5% oil-enriched Artemia, the impact of the nutritional value was significantly (p<0.05) reflected in increase in the growth of the larvae compared to those larvae fed with newly hatched wild type Artemia. Pre-feeding with wild type SC Or SMD1168H did not enhance the nutritional quality of Artemia nauplii. A similar enhancement of the nutritional value of Artemia nauplii for marine fish larvae (red seabream) was demonstrated by Watanabe et al. (1980) using algae and ωyeasts containing EFA (DHA/EPA). From our experiments, it is apparent that the nutritional value of Artemia is governed by its content of EFA, especially 20:5 (n-3) and 22:6 (n-3), and that the nutritional value of Anemia can be enhanced by enriching the Artemia nauplii through pre-feeding with EFA-rich yeast rVtg clone #6 or #6 in 5% oil diets. While similarly, in this study high levels of HUFA are present in recombinant yeast clone #6 in 5% oil microcapsule containing cod liver oil, a major advantage of microcapsulated diet is that the enriching oil is not in direct contact with culture water. This reduces the potential of water quality degradation and bacterial proliferation.

Some authors have concluded that DHA is superior to EPA as an essential fatty acid for fish larvae. Watanabe (1993) studied the larviculture of five marine fish (red sea bream, yellowtail, striped knifejaw, Japanese flounder and trout) and found that DHA was necessary in greater quantities than EPA, and that its presence in adequate quantities increased survival and growth. Similarly, Rodriguez et al. (1997) reported that gilthead sea bream larvae achieved best growth rate a DHA:EPA ratio of about 1:3. Ibeas et al. (1997) obtained maximal growth of gilthead sea bream larvae with a ratio of 2, while in the juvenile stage the best ratio was found to be 0.5. The highest DHA:EPA ratio found in the present study was 0.25 (Artemia fed with #6 5% fish oil).

In the larvae feeding experiments, #6 and #6 5% fish oil proved to be an excellent nutrient supplement, in terms of growth and survival. In contrast, the survival rate was poorest (at 26%) when the larvae were fed exclusively on baker's yeast S. cerevisiae. Similarly, by replacing 50% of #6 with S. cerevisiae in the fish diet, only small differences were observed in growth and survival of the tilapia larvae. The wild type baker's yeast and P. pastoris have low dietary EFA levels (20:5 n-3 and 22:6 n-3). Low dietary EFA levels can lead to lower growth and higher mortality in fish larviculture (Scott and Middleton, 1979; Watanabe et al., 1983; Caric et al., 1993).

Several studies have shown the beneficial effect of feeding (n-3) HUFA-enriched Artemia nauplii to several species of marine fish and crustaceans (Leger et al., 1986). However, no study has clearly indicated the requirements for (n-3) HUFA for one species or another. Furthermore, the n-3 HUFA requirements of larval fish are highly variable. Such differences could be explained by species-specific nutritional requirements, culture period or quality of larvae. The latter can be influenced by both the nutritional conditions during gonad formation (Lavens and Sorgelos, 1991) and the process used for spawning induction of broodstock (Lam 1991). Some investigators have proposed that the presence of (n-3) HUFA may not be important for young (newly hatched) larvae. Kraul (1993) reported that the first stage survival (0-9 days) of mahimahi larvae was not affected by the content of fatty acids in the feed and suggested that this is probably due to high levels of endogenous EPA and DHA stored in the larval yolk sac. Mourente et al., (1993) suggested that the presence of DHA in the diet of larval gilthead sea bream might not be necessary, because they could retain DHA from yolk. A high mortality of gilthead sea bream larvae was observed during the first 2-3 days of external feeding (Rodriguez et al., 1998). The high mortality obtained just after yolk adsorption was probably due to the transition from endogenous to exogenous feeding. Thus, a major advantage of the rVtg yeast clone is its constitutive production of the precursor yolk proteins, hence partially mimicking the natural yolk, which may be useful in buffering sudden switch of feed for the post-yolk absorption larvae.

In conclusion, the genetically-engineered Pichia pastoris rVtg yeast clone #6 used in this investigation promotes good growth and excellent survival in tilapia larvae. They are relatively simple, rapid and economical to prepare using readily available laboratory chemicals and equipment. The clone is robust, can be cultured over long term with neither loss in Vtg gene nor its ability to constitutively express the Vtg protein. The clone can be freeze-dried for prolonged storage. Therefore, large-scale production of yeast biomass represents a potential high quality substitute for fish-based nutrients in aquaculture feeds. Specifically, clone #6 enriched with cod liver oil could be a superior source for DHA and EPA enrichment for a wide variety of oviparous animals. We have demonstrated that the yeast clone could be used either directly as a high quality live-nutrient feed for fish larvae or indirectly via enrichment through Artemia nauplii, which is subsequently fed to the larvae, resulting in significant improvements in growth and survival. The subtle source of high PUFA and HUFA in clone #6 renders it a uniquely enriched nutrient package.

### OTHER PUBLICATIONS

A O A C. 1980. Official Methods of Analysis. Association of Official Analytical Chemists, Washington, D.C., 1018 pp.
Bradford M M. 1976. A rapid and sensitive method for the quantification of microgram quantities of protein using the principle of protein dye binding. Anal Biochem 72: 248-254.
Burnette W N. 1981. Western blotting: Electrophoretic transfer of proteins from sodium dodecyl sulfate-polyacrylamide gels to unmodified nitrocellulose and radiographic detection with antibody and radioiodinated protein A. Anal Biochem 112: 404-427.
Chen C M, Cheng W T K, Chang Y C, Chang T J, Chen H L. 2000. Growth enhancement of fowls by dietary administration of recombinant yeast cultures containing enriched growth hormone. Life Sciences 67: 2103-2115.
Clare J J, Rayment F B, Ballantine S P, Sreekrishna K, Romanos M A. 1991a. High level expression of tetanus toxin fragment C in Pichia pastoris strains expressing multiple tandem integrations of the gene. Biotechnology 9: 455-460.
Clare J J, Romanos M A A, Rayment F B, Rowedder J E, Smith M A, Payne M M, Sreekrishna K, Henwood C A. 1991b. Production of mouse epidermal growth factor in yeast: high level secretion using Pichia pastoris strains containing multiple gene copies. Gene 105: 205-212.
Cowey C B, Cho C Y. 1993. Nutritional requirements of fish. Proc Nutrition Soc 52: 417-426.
Cregg J M, Vedvick, T S, Raschke W C. 1993. Recent advances in the expression of foreign genes in Pichia pastoris. Bio/Technology 7: 160-164.
De Silva S S, Anderson T A. (Eds). 1995. In: “Fish Nutrition in Aquaculture” (Chapman & Hall, UK, Aquaculture Series I), p 41-102.
Fitzsimmons K, Circa A, Jimenez E B, Pereda D. 1999. Development of low-cost supplemental feeds for tilapia in pond and cage culture. In: K McElwee, D Burke, M Niles, and H Egna (Eds), Sixteenth Annual Technical Reports. Pond Dynamics/Aquaculture CRSP, Oregon State University, Corvallis, Oreg., pp 57-63.
Folch J, Lees M, Sloane-Stanley G H. 1957. A simple method for the isolation and purification of total lipids from animal tissues. J Biol Chem 226: 497-509.
Guo X, Ota Y. 2000. Incorporation of eicosapentaenoic and docosahexaenoic acids by a yeast (F0726A). J App; Microbiol 89: 107-115.
Hollenberg C P, Gellisen G. 1997. Production of recombinant protein by methylotrophic yeasts. Curr Opin Biotech 8: 554-560.
Higgins D R, Cregg J M (Eds.) 1998. Pichia protocols. In: “Methods in Molecular Biology”, vol. 103. Human Press, Totowa, N.J.
Li Z J, Xiong F, Lin Q S, d'Anjou M, Daugulis A J, Yang D S C, Hew C L. 2001. Low temperature increases the yield of biologically active herring antifreeze protein in Pichia pastoris. Prot Expr Purif 21: 438-445.
Li A K, Sadasivam, M and Ding J L. 2003. Receptor-ligand interaction: vitellogenin receptor and vitellogenin, implications on LDLR and Apo B/E. The first three ligand binding repeats of vitellogenin receptor interact with the N-terminal region of vitellogenin. J. Biol. Chem. 278: 2799-2806.
Lim E H, Ding J L, Lam T J. 1991. Estradiol-induced vitellogenin gene expression in a teleost fish Oreochromis aureus. Gen Comp Endocrinol 82: 206-214.
Lim E H, Teo B Y, Lam T J, Ding J L. 2001. Sequence analysis of a fish vitellogenin cDNA with a large phosvitin domain. Gene 277: 175-186.
Lochmann R. 2001. Practical diet development for broodstock of Colossoma macropomum and Piaractus brachypomus. In: A. Gupta, K. McElwee, D. Burke, J. Burright, X. Cummings, and H. Egna (Eds), Eighteenth Annual Technical Report. Pond Dymanics/Aquaculture CRSP, Oregon State University, Corvallis, Oreg., pp 65-66.
Melo A C A, Valle D, Machado E A, Salerno A P, Paiva-Silva G O, Silva N L C E, De Souza W, Masuda H. 2000. Synthesis of vitellogenin by the follicle cells of Rhodnius prolixus. Insect Biochem Mol Biol 30: 549-557.
Owen J M, Adron J W, Middleton C, Cowey C B. 1975. Elongation and desaturation of dietary fatty acids in turbot (Scophthalmzis maximus) and rainbow trout (Salmo gairdneri Rich). Lipids 10: 528-531.
Rainuzzo J R, Reitan K I, Olsen Y. 1997. The significance of lipids at early stages of marine fish: a review. Aquaculture. 155: 303-115.
Romanos M. 1995. Advances in the use of Pichia pastoris for high-level gene expression. Curr Opin Biotechnol. 6: 527-533.
Sanderson G W, Jolly S O. 1994. The value of Phaffia yeast as a feed ingredient for salmonid fish. Aquaculture. 124: 193-200.
Sappington T W, Raikhel A S. 1998. Mini review. Molecular characteristics of insect vitellogenins and vitellogenin receptors. Insect Biochemistry and Molecular Biology. 28: 277-300.
Sargent J R, Henderson R J, Tocher D R. 1989. The lipids. In: Fish Nutrition, Halver J (Ed), 2^{nd} Ed. Academic Press, London, p 153-217.
Sivaloganathan B, Walford J, Ip Y K, Lam T J. 1998. Free amino acid and energy metabolism in eggs and larvae of seabass, Lates calcarifer. Marine Biology 131: 695-702.
Su H M, Su M S, Liao I C. 1994. Fatty acid composition of the rotifer Brachionus plicatilis fed selected microalgae or yeast alone or in combination with various oils. In: LM Chou et al. (eds), The Third Asian Fisheries Forum. Asian Fisheries Society, Manila: 207-210.
Su H M, Su M S, Liao I C. 1997. Preliminary results of providing various combinations of live foods to grouper (Epinephelus coioides) larvae. Hydrobiologia 358: 301-304.
Tan N S, Ding J L, Ho B. 2002. Engineering a novel secretion signal for cross-host recombinant protein expression. Protein Engineering 15: 337-345.
Vassileva A, Chugh D A, Swaminathan S, Khanna N. 2001. Effect of copy number on the expression levels of hepatitis B surface antigen in the methylotrophic yeast Pichia pastoris. Prot Expr Purif 21(1): 71-80
Watanabe T. 1982. Lipid nutrition in Fish. Comp Biochem Physiol B 73: 3-15.
Waterham H R, Digan M E, Koutz P J, Lair S V, Cregg J M. 1997. Isolation of the Pichia pastoris glyceraldehyde-3-phosphate dehydrogenase gene and regulation and use of its promoter. Gene 186: 3744.

1-37. (canceled)

38. An expression vector comprising a vitellogenin gene operably linked to a promoter, wherein the promoter is functional in a eukaryotic host suitable for use as a feed or feed additive.

39. The expression vector according to claim 38 wherein the promoter is functional in yeast.

40. The expression vector according to claim 39 wherein the promoter is a constitutive promoter.

41. The expression vector according to claim 40 wherein the promoter is a yeast glyceraldehyde-3-phosphate dehydrogenase (GAP) promoter.

42. The expression vector according to claim 41 comprising SEQ ID NO: 1.

43. The expression vector according to claim 42 which is Vtg (-SS)/pGAPZA, Vtg (VTGSS)/pGAPZA or Vtg (ÿSS)/pGAPZÿC.

44. A transgenic eukaryotic host suitable for use as a feed or feed additive comprising the expression vector according to claim 38.

45. A transgenic yeast comprising an expression vector wherein the expression vector comprises a vitellogenin gene operably linked to a promoter functional in yeast.

46. The transgenic yeast according to claim 45 wherein multiple copies of the expression vector has integrated into the yeast genome.

47. The transgenic yeast according to claim 45 wherein the promoter is yeast GAP promoter.

48. The transgenic yeast according to claim 47 comprising SEQ ID NO: 1.

49. The transgenic yeast according to claim 48 wherein the yeast is Pichia pastoris.

50. The transgenic yeast according to claim 49 wherein vitellogenin protein is expressed intracellularly.

51. The transgenic yeast according to claim 50 wherein yeast is protease deficient.

52. The transgenic yeast according to claim 51 wherein the amino acid and lipid contents are increased.

53. The transgenic yeast according to claim 52 wherein the level of polyunsaturated fatty acids is increased.

54-74. (canceled)


SEQUENCE LISTING (24 sequences)

SEQ ID NO: 1 - DNA, 5511 bp, Oreochromis aureus
  vitellogenin (Vtg1) precursor mRNA, egg yolk protein precursor mRNA
acatccaccagccatgagggtgcttgtactagctcttgctgtggctctcgcagtgggggaccagtccaacttggccccaggattcgcctctgttaagacctacatgtacaaatatgaagcggttcttatgggcggcctgcctgaagagggcctggctcgagctggggttaaaatccggggcaaagttttgatcagtgcaacaagtgccaacgactacattctgaagcttgtagaccctcagttgctggagtacagtggcatctggcccaaagatcctttccatccagccaccaagctcaccacagccctggctactcagctctcgacaccgatcaagtttgagtatacaaacggcgttgtcgggagactggctgcacctcctggggtctccacaacagtgctgaatatctacaggggaatcatcaacctcctgcagctgaatgtaaagaagacacagaacgtctacgagatgcaagagtctggagctcatggtgtgtgcaagaccaactatgtgatcagggaggacgcgagagccgaacgcattcatctgaccaagaccaaggacctgaaccactgccaggagaaaatcatgaaggccatcggcttggaacacgtagagaaatgccatgattgtgaagctagaggaaagagcctgaagggaactgcttcctataactacatcatgaagccagcacccagtggttctctgattatggaggctgtcgctagagaggtcatcgaattctcacctttcaacattttgaatggcgctgctcagatggagtctaagcaaattctgaccttcctggatatcgagaacacccctgtggatcatgccagatacacctatgttcaccgcggatccctgcagtatgagcatggcagcgagattctccagacacccatccatcttctgagggtcacccatgccgaggctcagattgtcagcactctgaaccacctggtagcctccaacgtggccaaggtccatgaagatgcccctctgaagtttgttgagctcatccaggtgatgcgtgtggccagatttgagactattgagtccctctgggctcagtttaaatctagacctgatcacaggtactggttactgaatgctgtcccccacattcgcactcacgctgcgcttaagttcctcattgagaagctccttgctaatgagttaagtgagactgaagctgctatggctctcttggaatgtctgcactctgtgacagctgaccagaaaaccattgaacttgtcagaagcctggctgagaaccacagagtgaaacgtaacgctgtgctcaacgagattgtgatgctgggctggggcactgtaatttccaggttctgtaaagcgcagccatcttgctcatctgatcttgtgacacctgtacatagacaagttgcagaggctgttgaaactggtgacatcgatcagctcactgtcacgctcaaatgcctggataacgctggacatcctgctagcattaagacaatcatgaagttcctgcctggctttggcagtgctgctgcccgagtcccactcaaagttcaggttgacgctgttctagccctgaggagaattgcaaagagggaacccaagatggtccaggaaatagctgctcagttgctcatggaaaagcatctccatgcagaactgcgtatggttgctgccatggtgctctttgagactaaactccccgtgggtctagcagctagcatttccacagccttgatcaaagaaaagaacctgcaggtcgttagctttgtctactcttacatgaaggccatggccaagaccacatcccctgaccacgtttctgttgctgcagcatgtaatgttgccttgaggttcctcaaccccaaattaggcagactgaacttccgctacagccgagccttccatgtggatacctataacaatgcctggatgatgggtgctgccgccagtgccgtcttaattaacgacgctgcaaccgtgttaccaagaatgattatggccaaagcccgtacttacatggccggagcttatgttgatgcttttgaggttggagtgaggactgagggaatccaggaggctcttttgaaaagacgacatgaaaattctgagaatgcagacaggatcaccaagattaaacaagccatgagagctctttctgagtggagggctaatccttcgagccaggccctggcctctatgtatgtgaaggtcttcggacaagaaattgcatttgccaacattgacaaatccaaggttgaccagcttatccagtttgccagtggacctttgagaaacgtattcagagatgctgtgaattctgtgctgtctggttatgcaacacattttgctaaaccaatgctgctcggtgagctccgtctcatccttcccaccactgttgggttgcccatggagatcagcctcattacatccgctgtgactgctgcatctgttgacgtccaagccactgtgtcaccacctctgcctgtcaactaccgagtttcccagcttctggagtccgatatccaactgagggctacagttgctccaagtcttgccatgcagacctatgcattcatgggtgtgaacaccgccttaatccaggctgcagtgatgacaaaagccaaagtttacacagctgttcctgcacagataaaagcaaggattgacattgttaagggcaacttgaaggttgagttcctgtctctccagggcattaacacaattgcatctgcacatgcggagacggttgccattgcaagaaatgtggaagacctcccagccgcaagaagcacaccactgatctcatctgaaactgcatcacaactttcaaaggcctctctcaactcaaagatctccaggatggcatcctctgtgactggtggcatgtctgcgtcatctgaaatcattcctgctgacctgccaagtaagattgggaggaaaatgaaactccctaaaacctacaggaagaaaatccgtgcttcaagcagaatgctaggattcaaggcctacgctgagattgaatctcacaatgccgcctacatcagagactgccctctctacgctctgatcggaaagcatgctgcttctgttaggattgctccagcttctggaccagtcattgagaagattgaagttgagattcaggtcggagataaagcagcagaaaatatgattaaagcgattgacatgagcgaagaggaggaagctcttgaggataagaatgtcctcttgaaaatcaagaaaatactggcacctggtctcaagaacaccacatcatcttcctccagctcctccagctcctcttcatccagctctagctccaacaagtcttcttcatccagttcccgctccagcagctcccagtcatccagctctcgttcccataggtctcgctccagaaagtcccagtctagcagctctcagtcaagccgctctccctcaagctcttcctcctcttcctcctcttcatcatccagatcttcttccaggtcatcttccagatcatcttccagatcttcttctaggtcctcctctcgctccagaactaagatggctgacattgttgctcctattatcacgacgtccaccagagtgagcagttcctccagtcgatcagcctctaacagctcctccagcagtgcttcatacttgctcagctcatctaaaagaagaagcagaagcagaagcagcagcagcagtagcagcagcagcagcagcagcagcagtagcagcagcagcagcagcagcagtagcaagaacagcaagcgcagcaagagcagcaacagcaagtcatcaagctctaggtcctctcggcgcagtgctcagtctaagcaacaactgcttgccttgaagttcagaaagaaccacgtccacaggcatgccatctccacacagcgcggcagcagtcacagcagtgcccgcagcttcgattccatctacaataaggccaagtacctcgctaacacactcactcctgccatgtccattgcaatccgtgccgtgagagtcgaccacaaggtccagggataccagctagcagcttacctggacaaacagaccaatagactgcagttgatttttgccagagtcgctgagaaggacaactggagaatctgtgccgacattgtgcagctgagttcgcacaagctgatggccaagactgcctggggtgctgaatgcaagcaatactccaccatgattgtagctgaaactggtcttttgggtcatgagcccgcagcccgcttgaagctgacctgggacaaactgccaggaagcataaagcactacgcaaagagggcgttgaaatccattgtccctattgctcaagaatatggagtaaactacgcaaaggccaagaatcctcgtaatcaaatcaaactgactgtagctgttgctactgagacaagcatgaatattgtgctgaacacaccaaaggcaatcgtttacaagcgtggggtgtgtctacctgttgctttaccaattggaaacactgctgccgagctgcaagcgacccgggacaactgggctgacaagatgtcctatttggttaccaaagctaacgcagttgaatgctccctcatcaacaacacactgaccacattcaacaacaggaaagctagagatgagctgccacactcgtgctaccaggtcttggctcaggattgcacaccagaactcaaattcatggttctgctgaagaaagaccaaatacaggatcagaaccagatcaatgttaagatttcagacatcgatgtggacatgtatcggaagaacaacgccattgcggtgatggttaacggagttgaaatccctaacagcaacctgccatacctgcatccatcaggtaacatacatataagacagtcaaatgaaggcattactctcaatgcacccagccatggtcttcaggaggtcttccttggcttcaacgagctgagggttaaagttgcagactggatgaaaggaaagacttgtggtgcctgtggaacggcaagcggaaatgtcggagacgagtaccgcacacccagtgaacaggtgaccaaggatgccatcagctacgcccactcctgggttctgtcttcaaacacctgccgtgatccctccgagtgttccatcaagcaggaatctgtgaagctggagaagcgggtgatctttgaaggtgtggagtccaaatgctactctgttgagcccgtgctgcagtgcctgcccggctgtatcccagtgagaaccactaccgtcaacgttggctttcactgcctgcccagtgacacaactgtggaccgttctggtctgagcagcttctttgagaagagcatcgacctgagggatactgcagaagcccacctggcctgtcgctgcactcctcagtgtgcttaatctcattgccttcggtcacatgacattttgttttctgttttaggcaggtgttgttaattagctttggctcaacttgtaaacttaaataaattggaaaagcatgtgaagcaaaaaaaaaaaaaaaaaaaaaa

SEQ ID NO: 2 - protein, 1788 aa, Oreochromis aureus
  vitellogenin (Vtg1) precursor mRNA, egg yolk protein precursor mRNA
MRVLVLALAVALAVGDQSNLAPGFASVKTYMYKYEAVLMGGLPEEGLARAGVKIRGKVLISATSANDYILKLVDPQLLEYSGIWPKDPFHPATKLTTALATQLSTPIKFEYTNGVVGRLAAPPGVSTTVLNIYRGIINLLQLNVKKTQNVYEMQESGAHGVCKTNYVIREDARAERIHLTKTKDLNHCQEKIMKAIGLEHVEKCHDCEARGKSLKGTASYNYIMKPAPSGSLIMEAVAREVIEFSPFNILNGAAQMESKQILTFLDIENTPVDHARYTYVHRGSLQYEHGSEILQTPIHLLRVTHAEAQIVSTLNHLVASNVAKVHEDAPLKFVELIQVMRVARFETIESLWAQFKSRPDHRYWLLNAVPHIRTHAALKFLIEKLLANELSETEAAMALLECLHSVTADQKTIELVRSLAENHRVKRNAVLNEIVMLGWGTVISRFCKAQPSCSSDLVTPVHRQVAEAVETGDIDQLTVTLKCLDNAGHPASIKTIMKFLPGFGSAAARVPLKVQVDAVLALRRIAKREPKMVQEIAAQLLMEKHLHAELRMVAAMVLFETKLPVGLAASISTALIKEKNLQVVSFVYSYMKAMAKTTSPDHVSVAAACNVALRFLNPKLGRLNFRYSRAFHVDTYNNAWMMGAAASAVLINDAATVLPRMIMAKARTYMAGAYVDAFEVGVRTEGIQEALLKRRHENSENADRITKIKQAMRALSEWRANPSSQALASMYVKVFGQEIAFANIDKSKVDQLIQFASGPLRNVFRDAVNSVLSGYATHFAKPMLLGELRLILPTTVGLPMEISLITSAVTAASVDVQATVSPPLPVNYRVSQLLESDIQLRATVAPSLAMQTYAFMGVNTALIQAAVMTKAKVYTAVPAQIKARIDIVKGNLKVEFLSLQGINTIASAHAETVAIARNVEDLPAARSTPLISSETASQLSKASLNSKISRMASSVTGGMSASSEIIPADLPSKIGRKMKLPKTYRKKIRASSRMLGFKAYAEIESHNAAYIRDCPLYALIGKHAASVRIAPASGPVIEKIEVEIQVGDKAAENMIKAIDMSEEEEALEDKNVLLKIKKILAPGLKNTTSSSSSSSSSSSSSSSSNKSSSSSSRSSSSQSSSSRSHRSRSRKSQSSSSQSSRSPSSSSSSSSSSSSRSSSRSSSRSSSRSSSRSSSRSRTKMADIVAPIITTSTRVSSSSSRSASNSSSSSASYLLSSSKRRSRSRSSSSSSSSSSSSSSSSSSSSSSSKNSKRSKSSNSKSSSSRSSRRSAQSKQQLLALKFRKNHVHRHAISTQRGSSHSSARSFDSIYNKAKYLANTLTPAMSIAIRAVRVDHKVQGYQLAAYLDKQTNRLQLIFARVAEKDNWRICADIVQLSSHKLMAKTAWGAECKQYSTMIVAETGLLGHEPAARLKLTWDKLPGSIKHYAKRALKSIVPIAQEYGVNYAKAKNPRNQIKLTVAVATETSMNIVLNTPKAIVYKRGVCLPVALPIGNTAAELQATRDNWADKMSYLVTKANAVECSLINNTLTTFNNRKARDELPHSCYQVLAQDCTPELKFMVLLKKDQIQDQNQINVKISDIDVDMYRKNNAIAVMVNGVEIPNSNLPYLHPSGNIHIRQSNEGITLNAPSHGLQEVFLGFNELRVKVADWMKGKTCGACGTASGNVGDEYRTPSEQVTKDAISYAHSWVLSSNTCRDPSECSIKQESVKLEKRVIFEGVESKCYSVEPVLQCLPGCIPVRTTTVNVGFHCLPSDTTVDRSGLSSFFEKSIDLRDTAEAHLACRCTPQCA

SEQ ID NO: 3 - protein, 1782 aa, Bombyx mori
  domestic silkworm vitellogenin
MKLFVLAAIIAAVSSDRFSSQSQSTGGQTYPSPWQVGKQYRYEVTSRTLAHLQEGPSSGSAFKAQFTIRVKSPGRLQAKLENPQHGNFNEQLPDPRELPVDLKYQPTPNIDKVFEIEIDGGRIVSLDFPTSVPVPQENLIKGLISALQLDTSAHRVIHDSQNNYDREQQQGLFRKMETDVTGDCETLYTVSPVASEWRRELPKFANEQDPVEVTKSTNYGHCHHRVAYHFGVPVGAEWTGTAHKTQEQQLIGRATYSRILTGKEGPIYKAETTSTVHVHPHLYGKQKAEVYSHVHMELISVDQDSGAEWPRAGAMRPAQSILYSLSTKQMTKHYESSSSSSSSESHEFNFPEQHEHPHQSNQRSRRSYMRSKLVTVHKVLKKRNSESSSGSSSSSADSSSTYINDDIPDIDEPAYAALYMSPQPHADKKQNAMNAQKILQDIAQQLQNPNNMPKSDFLSKFNILVRLIASMSTEQLSQTSRSIETAKTSNNIIKSDMWMIFRDGVTQAGTLPAFKQIQSWIENKKIQEEEAAQVVVALPRTLRYPTKQIMTQFFNFARSPAVKDQMFLNSSALMAATKLINLGQVNNYTAHSYYPTHMYGRLTHKHDAFVLEEILPTLAADLKATVEYKDSTKAQVYIQAIGNLGHREILKVFAPYLEGKVEISTYLRTHIVKNLKTLAKLRDRHVRAVLFSILRNTAEPYPVRVAAIQSIFISHPTGEMMQAMAEMTHNDPSVEVRAVLKSAILSAAELQHPRNFYLSRTAQAARYLVTNEEFGYQHSFKFIDDSYDEDNDIGTFVISHIGSEDSLLPKDFKIVTNSKGGAWERNTIEASFSSAERFLDYLRDSVFAPHPKFDRAHKYSAEKIAKLLNIKNDEEEPLEASFYVDFMNNQRLFSFSESDLQQLSQYISEYMKKVESGAEKHYTKVYNQDQVSIMFPVASGMPFIFKYKEPAVIHFQSKLKGKFSFPSKDNKYYEANMIKDVQFTYAINIDGNVGFMDTLSNQYSSVGVVNKLQFNIPFKFGIEIKSGLIKFRVEPLHPDQDQTLVHYSVWPYSASQKKDSLVAISQDPATKIVERRSKVFSVDSKYGQSTHAVIYAQGYTYSSDWRNFGAKFTSRDYFTNLASLLTQEDIALTHFNLKHLCKQSQSKALTITAYYDEYYNQQNSGILTDATDRNDLSPNSETRRAEMVKLVSAGINKARVRVVDLSASFEGSQDQNYVLTGTWGDSPVDSKVQGMLFAGTKSATQGNQQINAVFATTKPEIHSLSFSKPLQSDLRAPFGMHFKYGQSGEIRVSGSFDRTKKYTTELENHPLAKQCSQQTTLNNFYQDSCHKAIVMAHAPDHVEFSVSFQDMSPQYRNFSYHTYRLYEYLGYWYTEANPLKLTQNGKMDFKIDFSYFDRTYTVDIASPSGEARMRDMPIATMAPGALSFYQPLKAYELVANYFTGHQYQPYCSIDGTRIHTFSNRSYEYPLSRSWHVVMQDESTQRGNWHELAILSRRQQRDQQEIYISYKSESGQDLEIEIQPASGDSAYQVKVTTNTKKITDDDLTMYWDDVKEQPFLQYHTHKDGVLVINIEDDRIRAIYDGQRFVVFTQDYRNSTRGICGRMSGEQRDDYLTPEGLVDKPELYAAAYSLNEENSDPKTQELKALATQQAYYPEYKYTSILRSDPTWQEESQSCGEDQWQSETVYKSRSYDKHKGACEVRQQVQFYENHGDICITTSRVPSCQSHCRAGDYKIQHVQVTCKSKLDHDFRMYKEQIKKGQNPEVSGIPSVKQFKVPVTCQP

SEQ ID NO: 4 - protein, 1747 aa, Lymantria dispar
  gypsy moth vitellogenin
MRLLLISAFIAVVSSSPLTEPQPVHQQSPWQNGKQYLYEVNSHTLSHFYEGTSSGNAFKANFIIRVTAQGLLQAKLEEPRRAQFHQKLDNKMQIPADLKYTNVVNLDQPFEISFDGGRILSLRLPEVLSVANENLLKGLISTLQVDLTNYRLVNRSQDSYDKNSQQGLFSKFETDVTGDCETVYSVSPVNAEWRREVPHFASDSAPIQITKSKNYNRCHQCIIYNFGVPRGALWTGIAHGNTEKQFISHTTVSRILVGHQGIYNTETTSSVNVHPHIHGRQKAEVHSYIQMRLKSIEENPGTNWQKPEATRHVKSLLHSMLTKQVQNSYQASSESNEETEGSTYTAQQPYEAYDGCYYESSSYLDSSNFVNNEQLKYNVPDYAAILLHTQPHVDKKQNAMSAQKLLQDLAQQLQNPSTMPQNEFLTKFNILVRLISTMTTDQLAQFSRGIEVGKASNKIAKVDMWFIFRDAVIQAGTQPAFQLIETWIKTKKIQGFLAAEVISSLAKTLRHLTKDTMKQFFELALCPEVLEQKYLNSSALIAAAEFIRLGSIDYEHSYPYYPPYVHGRFPLKNDRFIIENILPRLSQLLKQAVEQEDYGKTLVIVKAIGTLGHREILKVFTPYLEGQIKVSTYLRVQMILALKPLAEQKDRYVRSALFSILMNTAEPYEVRVAAAMILFLNEPTTDMLRVMAQLTNDDPSIHVRAVLKSSIETAATLEGPKYWHLAKAAQSVKELVTSEDFGYAYSAAWFFAKNFDSEEVTRYRLANYIGSDGSLIPNLFSLTWNNRFYGRGAENTVGFTSSNVQDIFDYIKQLILQPRKSTTEHQFSADKIAKLLNIKQDPLQPIESSIYYKIFDKEFFWPLDQPNIKQLVNDVFGFVKELENGVQSHYTKVFYSKQVSVIFPIATGEPFVFEYKEPVVVHLQTKLSGKINYPPTSKDISSNIQTEIQFTFARNMEGSVGFIDTNTNQLANIGVVKKYQINLPVKINVIGQSGKFQVKLEPLHTDQDLILLHHSVWPYSAYQQTNTTTTPSHDLKTKLIKRLKKVAEMNKELGQQFNLLFKYHGFSSSNTYRNAGDFVQDALDFSDFMSLNDLAETHYEFTYSGKQSKTKAVTFTAAYDVSHNLNSTGKFGEAHRFSDVTPNSAPRREEIVKRVSSGIKTATARVIDFSASFEGLQKLEYAVTAAVAGSMVDLKTQFAVFMGSQSDNGQINAVFKLQKPQMAPLDFHKALNSAVKYLIEADVTYGENSNINFKGHTERSQEYAEQLKNSLWANQCAQENAQQNKFQLGCHNVLIESHAPDRFKASITYKHIPAAHTALLDSYIQGLWSKGFEYNPSKRLPVGQIELEANASYVDQTANVAWTWWNGQVRFNNLPNTYITPALTTAYQPIGIEDSWTHFANSYSYHQYEPFCTVDGTKVKTFSNRDYNVTLPEIWTVLMHAQTNWEELVVLAKRPNEAKAKREIGKIGKLDLYISHKTATGKHLEVNIPYSAANNKANVKVETNAQLVADGDLTTYWDDVAETPLLQYSNHPDRVLLLHLSDGLHLLFDGKRGIFTTSQYRNITRGICGQNSGDPLDDYKTPLGIVDHSQHFGAAFTLDLEKTNSQIQQWKKIAQETAYQPKLTHTVILRFDEEWKIAGEQKGLEWGSQKVYRSRSYQKQRGPCQVQNQVQYHENHGEICITTTPISACQSHCHSSNYQVQAVQAVCKGKKDPEFRMYKDQIHQGQNPQVTGVPKVEQYRVPTTCTE

SEQ ID NO: 5 - protein, 1872 aa, Athalia rosae
  coleseed sawfly vitellogenin
MWSPLLLCLLVGIASADQHAWKTGQEYTYQVRGRTLAALHQVADQYTGIALKAQLKCQPKGSDALSCNIQRPQVAEIHAQLPGGWDSPLPEKKTNYQKFELSEKPFEIKFKNGVIDDLVVEKSLPTWQVNIIKSIASQLQVDTQGENAMKSRYNQEPEGDQAYATYKAMEDTVTGECEVLYDISPLPEYVLQTKPELAPVPQLKGSGQIIDIVKTSNFSNCEQRMGYHFGITGETDWEAGSNSMGDFLAKSSVSRVVISGSLDRYTIQSSVTTNKIVVSPNLKYSQKGMVVSRLNITLDSVNAASGSPRSPSEPESTGNLVYSYNSPFEANNKRRPHNNNDDDSSSSSSSSSSSSSSSSSSSSSSSSEEDQQRGNSENKNNNQHGHRQARAANQRGQGNQDSDSSSSSSSSSSSSSSSDSSSSSSSESDSLSSSEEYWQSRPTLTDAPEAPMLPLFIGYKGSAAQQSSQVNPVSVAKKLASQIGEELQKPSNIPKQNTLEKFSHLAALLRTMSAKQIEQAAHDMYAPAKENASKSESAKNAAWKAFRDAVAQAGTGPAFSVIKNWIQTNKIRNNQAADLIAVLPSTTRYPTAEYMDAFFELATSPEVTKQAPLNNTAILAFADLVRESQVNNASAHGRYPVHSFGRLSPKNNPAVQGQWIPYIAKQLKSAIRAADSQKIQVYIRALGNVGHPKMLSVFEPYLEGQEDSSDFQRLSMVLSMGNLARLYPKQARAVLYKIYQNTGEAYQVRTAAVFQLMRTNPPPSMLQRMAEFTNQDTSKQVNAAVKSIIETAAKLDDAELAHNARAARDLLSTKNMGLQYSRGWLHDYTSEAMSSGYQLNSYWIGSEDAIAPQAVFATAAFNKGGFQSPASIYGAMVSSVDDLLDVLYDQFEDNDEANQKKNSKSSKFSPENILNALNIQSNDAEQLEGSFLFPALQGNRFYAFDNNTIEQLPKVVKQAAAALKHGQSFNYTKLYSNEELTVSFPTAMGLPFVYTMRTPSLVQVGGEIQARTYPEQYSGSNEEISIPEAVNASLDIHAVYSSKVIARIGFVNPANHQHHMAGYDKNFQVNLPLSAEMDIDLENSHVRVKLEPIQQNKEYNLFHYSSWPYTAQKDILSEKPVSQQKGYALISERGSEQNEWAFGQKSTGLALRFTSEGEQTEHQLRNWAWEMLQSEDPLAIAFSLDDEIQHSNYNLWLDGQHSSNEAVVLTASYANYTKGMGGDSDSQSGYQHPAGRKNSGGEDNQSSSTFVPAFQPSSQKPDSESRQQELLQKAASGIKDAYSQSIDFGAVFQGKNKAEYAATFAMANSPVDPKSRVVFYYGSNPAQQKNYQICLSAQSSRPKVPVMDFIKALKADPTTKFAADLKFGEQCQSGAQVQIQGQLEQTEGRREYLRRTPTGQQCAQQMQEGNKILPACRNVTARANFLNEGSLTVKFSHVTDYMKNVSARAYGLARYFGYHSAYENIVNPGNQKNGQIDFGYEISPDFQRLNVSMSAPGLDANITDLEIHPWARSIVAVDPVQSGARRFAREALAGQYNPTCVIDNNQASTFDNKTYPIELGNCWHVVMTTIPDDEQNGDDEIPEDMRVSVVCRDINEHQRECRLVLGEAEIEMYPNNDQAAIKVNGRAVQLSQEETYRYTSQNGEIQFEAYLKPSGVVYIHSEEYGVRALYDGERIKLILNNEYRDQIRGLCGTFNGEPATDFTAPQNCILKNPEHFAASYALTKDQCEGPAKENARRAQQAHCYREAVLFGNVVSDQDAGRSKNKDSKWENNKSRKSGSGGKKSGSCTKFATATIEENGKTCFSLRPVATCAAGCKATRKQETTVKMHCVQSSSSSQQLVHRIKQGANPDFSQKSHDKSSRIEVPESCVAQQ

SEQ ID NO: 6 - protein, 1807 aa, Pimpla nipponica
  parasitoid wasp vitellogenin
MWCPLFLVLLAGAATAEHLQAWKTDTEYQYAVRGRTLSALHDVADQYSGIIMRALLTIQPKSDGTLEAKVTKARYAQIHTKLPGGWNSEIPDHKLEMKQFPMSEKVFEIKMKHGVVRDLIVDKDVPTWEVNVLKSIVSQLQVDTQGENLMASKYNQEPEDEGVTAMFKTMEDTVGGRCEVLYDINPLPEFVLQKRPELVPMPDIRGDGEIIDIVKTKNYSNCEQRSGYHFGIPGTNKWEPSSGASGNFLSRSSVSRVIVTGNLKSFTIQSSVSTNKVILSPNFHENRKGMVSSRVNVTLVKIGAPSTGDWSTPANPESTGNLVYNYNNPFAGVGEDRRASRPQDNRNSEEKVRSLYNLYRRNRINDNDDDSSASDSSKSAKSLESNEEQLYWQPKPTLNEAPAMAMLPFFIGNHGKSIHKTDEIEPITMAKTLASQIGSDFQDPNSITDEQTLEKFTLLVRIIRTMSTQQIAEAERDLYQSNNEIDPHDESQSVRRGTWAAFRDAVAQAGTAPALVTITLWIKQKKIRGVEAASVVGVLAKTARTPTREYIDVFFELATMPETIHEPFLNTTALFTFSELVRYSQMDNPSSHTRYPVHTFGRFSDKINPEVFRMYVPYLAEKLKTSIEKNENAKAHTYIVSLGNIAHPKILAVFEPYLEGKMPASTFQRLLMVISLNKLATLKPKLARGVFYRIYKNTGEAHQLRCAAVTALMSTNPPASMLQRMAEFTNEDHSKHVNAAVKSAIESASELETPQWQELAENARNAKPLLNKESYGFEYSKLYLTDFIEREMNVAYQAQASFISSDDSYVPEALFVKARAIFGGFTFPRTSAGAMVSSAKDLLSAFEDAFKGEKTSHKHSHVEFSPENVAKLLKIKSEPNEDVEGAFMLQSAYNNKFMSLDKNTLKNLPELSSKALKALKDGHHFNTARIESYEMTMSFPMESGFPFVYTMKVPSMLKLAGSVKGDSNEDSLDGHVHLRAVYSVQFQSKLGFVTPFEHQHYMAGINKNYQAYVPLRVNVNYEDSDKRVTLKIQPIEQNEKFKLWHHSVVPFTSRHDILKMSPVLKDKETQKIITEEVSKNEYNFGSDKHGVKFTVLAEADNDNSPLNFEDAKWERNPLTQLFHLFVEQPVYHKVDIFMQPSKSSEQSIILSTAFETLQDDTTKYDEDRSKHIKAEAPKVENKKLNDPQRRRKLLKEAAKGIAAADAFAVDIGLEYPGQAPVQAFATLAFASSEVDEKSRGLVYWRLTDAGNDFEFESCTSLEARSPRTSDFGKDMLDQPEFRSRKFDIDLRYGQTCEKGYKVEIQGSQEQTEKYRDTIKRLPTAIECIRAAKKGMKSLPACHDVSVKMTMLDKTRITIKYDKDSREMYQLFDDYLDSMSDADDDLKSSKKRLNAVKDDDKAYDDTVKIAVDLSPNDNNGRFSYESDFVKLEARDVPINGIGPLVNVHPDLETAERLDMDMSDEPVRQRICVLDSHSATTFDESTYPINLGKCWHVVMTTFPKTNKNSGSAEPIDEDMALSILVRDAGDKKKDIKVTLGDKELQFSHADSKNKVTLDGKKVDLSEKRSYRHKNGKDIDFEVLQRPDGTLGLVSSKYDIDAWYDGQRVQIKASGKYRSDIRGLCGNFDGEPDNDFTSPKDCVLLKPEEFAASYALTTKDCHGSALEHARKASQAVCSQKSPRPGNVVSDRDAGRKYSENSNWGYHSRQNSDDEQGKNDSSDHKRCNTLRTKVIEEEDQICFSLRPLPTCAEGCTANRTKPKVMPMHCMPKNIAAERMADRIKQGANPDFSQKSYTKKNGFDIPVGCHAA

SEQ ID NO: 7 - protein, 1876 aa, Riptortus clavatus
  bean bug vitellogenin, bean bug yolk protein
MWSPVIICLLVGLASADNPWKAGTKYTYQIQGRTLTGLHQVADQYTGVMIQGQLTVRAESDNKATAVIENAKYADLQANLSRGWDSEIQENELQYNELPISKDPFEIHFRNGVIEKMVVNKDIPQWQLNFYKSIASQFQVDTQGKNLKDSKINLVPSGQSGEPMGVYKTMEDSVNGIYETLYDISVLPEYILQARPELAPLPHLKGDGEFIEVVKTRNFSNSNQHVIYRFGSSGHEGWEAGTNQMGDFLSRSSQSRVILSGKLNRYTIQSAVTTEKVLVAPHVYNNQKGIVVSSMNITLQQATGSQGSPQSVSNPREVKSLVYEYESQGNSENLQSSQQSSQQSSSEESSSSSSSSSSSSSEENMNSMKTNQQMKEDRAKRIRRSLEEQQSSINRKGKNARSQADSSSSSSSSSSSSSSSSSSSSDSSSSVSSSEENLPRQPRSIEQAPGSELQAGYYMGSMGQQKQNVPEQIKSLAKKVGQWVQNPGQIPEENRLPLFSMLTRMVQNANSQQLEQVTQSLYHQQSKNQQQSDAERQQYQSWAAFRDAVAQAGTGPALVTIKQWIQSKKVQGEEAAQLLAVLPYTARYPNTEYMNYFFDMVTSEEIQRQRSLNTTALFAFTELSRKSQVDYDVAHNRYPVHLHAKNSQEHRQAVPQKYIPWLQERLERAVSRGDSIKAQVYIRALGNTAHPKILAVFEPYLEGKSQISEFQRLSIIASLDEMTRTHPNLARDVLYRIYQNTAEKSEIRAAAVRQLMRTNPPAQMLQRMADFTNYDHSHQVNAAVKSAIESAAACTDSSVDQQLVNNARAAANYLTPKNYGVQYSKTHLKSHNVEGQNLQYQQHCSSIQSQDSSLPSSLMYEVQQSVGGYHRDPHKFYFMTSSSEQAINLMRQTIAQQSGSYRKRWSRNQHGSEQSYENILRSIHLHTDEPVQVEGNILVSGLGGKEFFTFDNHTIEQIPQFFSNINEKLQDKSYVQHTKYYNQYSLEIGFPSAMGLPFVYHLDVPTLASVSGKWSGQVELQQHQKEQNEKYSIPVKGQVHGDFEFVYSAQKSGLMGFVAPFSQNRYIAGLEKNIHAYVPVQAKVEYNPEENKVQASLQPLAQQKQEEQQVFQYSSNAYTTYQKSSDLTPYLQGENTQAVYAKAPRWYHNTYGQAATGYAFDLSYKTEHNSHSWASIYNALQCHDYISAFLYYDTPGTIQNDYISIKYNPQKSSPQEVNVALSFISNEYGNKGARMTSKFKKANIRDRSASSARDNLAVPSSEQQREQELLRNVEAEIQDAKSHLLDASVTFGGKNNNAGYSFTLAHANSPVSEKSRVLFYYNAQPVSSSENTKPFQAAFQLDIKDPQLPAFNYQDALNADSTSQVQGKINMGEQLESQITFKGKLERTQERKEYLQQHPLARACESQMQRRNYIQPACLNATLQANFHNRYQFVFNYQNVPAGVRRAFHSAYSMGRYALYQNNEENINTEQNQEGQIQIGVQFEENLRSVNLSIAAPGLSSNFSNVRVPGMAVPFVVYHPRYNTYQLLKSKLYQTEQPQATAVIDGNKATTFSNRTYPIDLGNCYHVFAMYAPQGQGENRQHESSISNIQQGYAVLVKETDSEHKEVKVILGSDVIELKSKSSGLSIQANGQNIHASEKQLTKWSNNNNYLEAYARPDAVVLSLPQQGIELTYDGARVQLSASSSYKGNVRGLAGTFDGQEANDFTTPNNRVLRNPLQFAATYALLDSECQGPAVQRQQQAQQSPSYERRVILGDVVNELEAGRQQQQNNKSKKNKANGSEERMKVQGSSCSKLMTQIVEENDKSCFSTTPQPACASHCRPTGKIQKIVDFHCVQASSSSRNWAQMIKNGANPDFSAKEKHRQILLQLPTGCVPKA

SEQ ID NO: 8 - protein, 2139 aa, Aedes aegypti
  yellow fever mosquito vitellogenin
MLAKLLLLALAGLTAAYQYENSFKGYNPGYKGYDAGYKGYGYDAGYKYNNQGYSYKNGFEYGYQNAYQAAFYKHRPNVTEFEFSSWMPNYEYVYNVTSKTMTALAELDDQWTGVFTRAYLVIRPKSRDYVVAYVKQPEYAVFNERLPHGYATKFYHDMFKFQPMPMSSKPFGIRYHKGAIKGLYVEKTIPNNEVNILKAWISQLQVDTRGANLMHSSKPIHPSKNEWNGHYKVMEPLVTGECETHYDVNLIPAYMIQAHKQWVPQGQLRGEDDQFIQVTKTQNFDRCDQRMGYHFGFTGYSDFRPNTNQMGNVASKSLVSYMYLTGNWYNFTIQSSSMINKVAIAPSLVNKEPALVYAQVNMTLNDVHPYDKVPMGPAEDLKVFVDLVYSYNMPSDKKNYVRPGNETSSSSSSSSSSSSSSSESSSSSSESVENPKISPVEQYKPLLDKVEKRGNRYRRDLNAIKEKKYYEAYKMDQYRLHRLNDTSSDSSSSDSSSSSSSESKEHRNGTSSYSSSSSSSSSSSSSESSSYSSSSSSSSESYSISSEEYYYQPTPANFSYAPEAPFLPFFTGYKGYNIFYARNVDAIRSVGKLVEEIASDLENPSNLPKANTMSKFNILTRAIRAMGYEDIYELAQKYFVSQKERQVAQFSDKKFSKRVDAWVTLRDAVAEAGTPSAFKLIFDFIKEKKLRGYEAATVIASLAQSIRYPTEHLLHEFFLLVTSDVVLHQEYLNATALFAYSNFVNQAHVSNRSAYNYYPVFSFGRLADADYKIIEHKIVPWFAHQLREAVNEGDSVKIQVYIRSLGNLGHPQILSVFEPYLEGTIQITDFQRLAIMVALDNLVIYYPSLARSVLYRAYQNTADVHEVRCAAVHLLMRTDPPADMLQRMAEFTHHDPSLYVRAAVKSAIETAALADDYDEDSKLALNAKAAINFLNPEDVSIQYSFNHIRDYALENLELSYRLHYGEIASNDHRYPSGLFYHLRQNFGGFKKYTSFYYLVSSMEAFFDIFKKQYNTKYFADYYKSADYSTNYYNFDKYSKYYKQYYYSKDSEYYQKFYGQKKDYYNDKEPFKFTAPRIAKLLNIDAEEAEQLEGQLLFKLFNGYFFTAFDNQTIENLPHKMRHLFENLEDGYAFDVTKFYQQQDVVLAWPLATGFPFIYTLKAPTVFKFEVDASAKTHPQVYKMPAGHPETENDDFFYMPQSINGSVDVNLLYHRMVDAKVGFVTPFDHQRYIAGYQKKLHGYLPFNVELGLDFVKDEYEFEFKFLEPKDDHLLFHMSSWPYTGYKDITDMRPIAENPNAKIVHDDNQSTKTMEHTFGQDMTGVALRFHAKYDFDLINFQQFWSLIQKNDFVSAVNYPFAYQPYEYHQFNLFYDSQRTHAKSFKFYAYQKFGAPSFEETGPKHPANRHSYSGNYYESNYAQPFVYSPGSQRRYEQFFRNAASGIRNSFVRYYDFGFEFYAPQYKSEFTFTTAFADSPVDKTSRQLYYFYASPMFPSQSYFKDIPFSGKQFQFCATATSEFPRVPYLKFSDFDKYYGDASQYFDFLYGESCQGGAHIAVKGKQKQTGKYREYLRFSDVAKACKEQMANGYYQFEECQQAIDQAYYYDFYDYAIEYKDVGSVAKNLTNKFYNYFQYAFYPYFESNFFYHGKSNYIKAEFEFAPYGDYYNASFFGPSYAFQVQNYPVFNDYSTYFPYFFKYTFFPRYQPYYMHRLPSHKPRNRPYYELSNYEQFAIFDRKPQYPSCSFSNDYFYTFDNKKYFYDMGECWHAVMYTVKPDYDFYAQQSHFYNSDFEYKYKNGFEEYEQFAALARRGSDNQLYFKFLFGDNYIEVFPNNGGVPFVKYNGRPYDISKSNIAHFEYKEGYPSFPFFYAFAYPNKDLEVSFFGGKLKFATDGYRARFFSDYSFYNNFVGLCGTNNGEYFDEFVTPDQCYMRKPEFFAASYAITGQNCTGPAKAFNYAYQQKAKQECVKREVYYGDIIYNQEYYHPRYRYYNHNVEESSSSSSSSSSDSSSSSSSSEFSSLSRSGSSSSSSSSEEQKEFHPHKQEHSMKECPVQHQHQFFEQGDRICFSLRPLPVCHSKCAATEKISKYFDVHCFEKDSTQAKKYKSEIGRGYTPDFKSFAPHKTYKFNYPKSCVYKAY

SEQ ID NO: 9 - protein, 1790 aa, Anthonomus grandis
  boll weevil vitellogenin
MWSTVALCLLVGLSYVSSSSPAWKDNTEYVYSVNGRTLTGLEETADQYSGVFLEAKLHLSIRPDGKLQGRISEPKFAQILSQLPDGWKSEIPDSQISYKQLQLSQKPFQLVLENGLIKRLIVEKDTLNWEANIIKSIVSQFQMDLQGENALQNPTSSFPTNEYMDAVFKTMEETVTGKTETIYDIHRLPEYLVQSQPWIAPQYKLKGEGDLIEVIKSKNYTNARDRPSYHYGFGEIEESEPTANKMGQFFIRQSNSRAILTGKPSRYIIQSTYTVNKIMVNPILKNKEMGSITSMVNVTLLEINNQQQQPEELSNPLDIGNLVYTYGQPKNNQVHSKLNENLMEDSSSEESSEQEMTHRRFRRSANSLTKQWRESSEEWNQQQQQPRPQLTRAPHSPLLPSMVGYHGKSIKENKDFDIRQNVENLVTEISDEIKQSEKTISKHTLDKYTILNTLVRLMDEDDIQFVAEQMYSQMKNGQQRYTWSIFRDSVAEAGTGPALLNIKKWIETKKIQKTEAAQVIGTLAQSTRFPTEEYMRKFFELATETQVRQQETLNQTCILSYTNLVHKVYINRNESHNQFPVHAFGSFYTKKGREFVKTTVIPHLKQELEKAISNADNNKIHVMIRALGNIGHKSILNVFQPYFEGEKQVSQFQRLMMVACMDRLADCYPHIARSVFYKIYQNTAELPEIRVVAVHQLIRANPPVEMLQRMAQYTNTDSQEEVNAAVKSVIESSCKLESSKHAELRKAAQSARPLLTKKQYGMEQSYINLRDYVAEQMGLELHVQRTSHSSAESSFPKIMKFQLHQHNHGMKQHILSTGGMISSIRELLNVLYRQTEVFQQEKSQRSQEQGKDNEWSSANIARLMNYERDEREQLEAIIYAQVEDVQKLWSFDNQTLEHLPEVIRQQEEIYRQGKDFSYVKLKQLNEMALSFPTEMGLPFLYTYDVPVLMKVEGKIRALANPAISRNNKLTKPEQISTEIKARVTCTGKTQSHLSFVTPFDHQIYMAGYDKNMYVSIPVNARLEMDVKSKEAKIEFEVEQQQQDSRLVHITSTPYTSRSDVMAISPVALRPNTYVIKSHRNNHRYFDFNFGKKETGLTFRGWGHHPEQSIGFNDLVSMWQSRGVAGVWEQLWDKCSTEYSEATISFIPSQSTTRKATFRINVDQKYQKQPETQSPEDLLTLNQLSSKLQKDEPKQRQQEIKKHVGSGINSALLSCSDISLEFEGDKKYEHVVGFAVAKSNADPKSRVMFYYKNKNENKQGALEIRSEIPNTNGLNLDDSLDTEPSTKYNMRLQYGNSENDAFEISAQAQLSRSQERKQYLINQDPLYHVCKEQMQQKNFQLPACQNMTIKANFLDHIKYQVQYQKLNWKLVETLEGMFKGLRVLYYPMTEIKSISSVGQNVVEGEVQFQPEDFRQVNVTVRNTDEETVFFNISLNNELLRTLLVPHPVFHAKCRFAGLMQGQQNYRPTCVIDQTTAQTFSNKTYSVNLDKEPTVVMQYVPKDARVNGQQSKSVEQLLRESIENYVVLVRQVAANQKEVIINLNHPRTQGKTVKIEMKPSEDRQKSARNPAAKVTIDGQEMHFDDKQIADKCDGYVQVYALPNGEVKLEVEDAFYLIYDGQRVKVTATGNKLRDSVYGLCGRFSQDKHEDFTVPSNCVTRDTRKFVESYQVEKGQQWRNSPSEQCIKKVLPLYTNVISNQNGSQMRTKLASGTVMKHRYIEENGEICFTIRPLPVCNTSVKQVVTKNVPVHCIQGTKTAYYYKSLIDQGGNPDFSRKSETRTARMEVAAQCN

SEQ ID NO: 10 - protein, 1862 aa, Blattella germanica
  German cockroach vitellogenin
MTWNALLCCLLVSAASAITPGWLPINSQLDYHVHGRTFSSLFQVANQYTGILYKARLSLDRNEDQLITGKVTEAQFSPVNTQFSSGWDESVPDEKLHWDVVPMSQQPFQIELNSRGEVRKLRVNKFVELWEINMIKAIISQLQVVVDEDKKVYRVFESTVTGRCEALYEVDHLYPTTYLNPWQWTQQHDTKLRIMKTHQFTNCRHNSAYKLHFNAFEYFHLKQHKPETFLSNSAVSRVIADGDNLKNFTFYSGETIHKIVLNPEIYNKQKGMLVSHINVTVERKGRELTVIDYELRNVGDLSYSTSLVKAHSMRNSASMDLSSSSMSSSSSSSSSSSSSSSSSSSSSSSEEHHSHNQKLSKKRQVPLPRPLFEANFDASSGLTTEQPVTFRPRRQLFQGQDMSEEETEQNPEIIPANLLPTYNLIHNTKQVDVDPVGVAVRLSKDIAADLQGEPRVGEDRHILPRFTILVRLLKQLKVSQIMEAARKLYKLENDHPNYMNWDTWRVYRDAVSQAGTWSALNSIQQFISSEMVEPKEASHLITVLPAAVSDKNKAYLHFLFEMTKDPVFKNMTYVNTSLVLAFSEVIHQVEMHQVRDLKIKSVYIPYLVQEFDDAVKENNSIKIQLYTHALGVTGNTHILHYLRPYIIQLKTITHHQRLFMVQSLERVVEHNPRKVIDLLLSLYLDQNEHADIRVEALFLLMKADPSIHVLKMVAELTHTESNNQVLSASQSAIKSAANVEGDIYSEMRRKAKAVEHLLSTRNMDVSYSKSYLYGYKSKKINYDSLYNLNYIGSEDSIYPKSMLLNIFTNNLGRINTHVQKGYMVSSMTDLWEAFHTIYKKDNGSPTDPKTLVKFVEGNLKYFNMGVQKFWAFDNTTFSNASAVIQEFLKTYKKPTNFNHTKLSSSSSITLTLPCAMGLPAYFKMNSPSLWKYNGEFSIQTDAKTDVPMSLENFMNITGSINLMFSQMYHAQLAFSTAFDNKEYISGLDRKVEVHVPVKFQINLDFKNHNGFIRIIPLFTDRDYDVLQWQTIPYTTIHNVPDFETVYMDQLFKLIHVRKTAHFEKKMGENTGIVFKVKYDTDQEFLDTKWFLDEFKVLQLFTGLNYDVPTKDIFYNNLTVYYDHEDTKNHAVSFTVTKEQSKFYETLNPVVQQNLKLSSGKKQKHRNVKSHRIRREYTEDENPAIPKDKQPNSHPRRQEYLSKSMALTGDATAVVLDMTLKFEGPAESYFTTTVSHATSLVNGSSNYLLFYDQHYYEEKKRNQFCLSWSVYKPQVPIMNIYSAFEFDPNSKVHAIMNIGKECENGGSAVANIDMLRLSEHLDYVKNLTVSKLCDHEMRTKRDHVLPACRNSTERASDLNRVHVDINYNLKQHETFKRRVYKVYDFVRTHLYPHVSEDVIVDNPAQFISANFTLKDNTRAFNVSIETPVLSVNATSVRLQSWQSEMLRMNPRTSFAKRFAKWALPLYYKPTCVVDSSYINTFDNFTYSAHHIVQNDAFYTILDIPQKFNMEYFKVAFKPTSPVPNMQREVLVFLRNAKIELKPNQGMPEVYVEGKRVDYNHHHSTDLNVSQDRIGYVYALPTKAAHIVFPSYEIEMFYDGSRIMIQASNMYRNFTKGLCGNMDGEFVNDVLTPWGCYAKDMALFVASYADNSNSEVRKIKATQNEQTCVPQFHQPLVSHQMRLSQVIKLADTSSSSESSSSSESHENNSSPSSESQVNKSKRQPNSRPRSSSSSSSSSSSESNESVLAKKIINNQIGPKPTLIPSQSPMTSDDKCMTQQPRHTYYENQFCVSEKPLDTCMPLICHATESYTIDVNFYCVPLGPAANHYMKLVKKGILPDLSRNRNGKRVVLPVEIPIQCEPVLN

SEQ ID NO: 11 - protein, 2587 aa, Marsupenaeus japonicus
  karuma prawn vitellogenin
  SIGNAL ()..(18): signal peptide
MTTSSLLFVLALVAGGLAAPWGADLPRCSTECPISGSPKLAYQPEKTYTYQYSGKSRVQLKGVDDGVSETEWAARVDLTWISPCDVAISFNNMKMDGARGPIAARTLERHPLVVAVVDGRVQHVCAHPDDEPWAINLKKGVASAFQNSIPSLSTVSSGMTVTETDVVGKCPTTYQIETEGEKVIVVKEKNHRHCQQRYPTPAETPAPWLKAPLPIEESKSECKQEITNGIYTSIMCHDKNIVRPAIGIYKYVEANQESTLHFISETTDTSAITAIPRGEMHIESLLYNHETMKDPELAPELDQLMKEICEKTKDTVEAEAAALVAKALHLLRRVPETVVVEIAQKVRQGHYCSDSAKLESIFLDAVAFLHESGAVKVMVQEILNGRATGGRLALYTAALYLTPRPNIEAVKALTPLFESPRPMPSLLLAAASMVNHYCRHTPHCHQEAPVERIAEILAAKVEGHCSPSIGVEEKEEALAIFKALGNMGVVTPAVTRAAAQCIEKEGLETSIRVAAAQAFRQANCDRPAVQKLVDIATRPTFETEVRIASYLAAIRCAEKEHLEQIIEKISEEENTQVRGFVLGHLINIQESTCPTKENLKYLLTNVVIPTDFEKDFRKFSRNVEMSYHAPAFGMGADLESNIIYAPGSFIPRAVNLNMKAAVDETHMDLAEIGARFEGIDSIIEELFGPEGYLRKATFGKIMQDITGFAEEKGLKVMEHIKQTLRTKRSIDSSVISDFFGKLYGEGRSHTHAEVFARIMGHEITYADVAESLKGVTADTLIETFFSFFEESLEQMKGLNLNTARTAQLYMDYSLPTIQGTPLKLKLAGTAVAGLKMEGDFNIAQILSDPGNLQTGIKLFPALSVQATGFVGFECRLTRVGIEMENTISSATGASINIRTTENKKIQMELEIPEKMELLNIKAETYLVKAVGKKLTKITPPTVRDVRVTHAACLNAVEPVLGIKVCYNINMPDVFRANGLPLGEPAIAKLYIEKADPSMRGYLMTAAIKNKKGNKFIKLNVEAAGATTPRRAEMTLSYTKEEGSHIVSAKLDSSSIAAGVWATLTNEEGHKAMETYVKFDYGQIAISRGIKLDMIVKEESAGKEFEVNVFSGRSRRFTPESHIVEAKFIKKTNGPEVNVDVICRTRNALAQYFDLNIEVGADFMEFSPEGVYPARYIPKVSILLPVALRKMEVHANTVAWKLASYIREGSQSGESRELISAFKLSKGRNDIIYVQATHKIEGTLPQNIVIENEATVEVGRSSYRAMYDIFYHPEKMGASVEVFRTAGNEKVAEMEAIYENTGEKYYTKFLVEAPGYIRPVRIEATAEEETGGRYALESAIKYGERTVFEVTGPVMARFTSKTAKLQANIKLSAMASEPYIIGANFVFGNKKQMIAMEIKEREEPVFGVEWKMVQESAEKTTLSIAFVLPALIENKVDAVITEDLVHVSFNNLVLPKTSYRRRVKGFADVNIGEKRANVEFSWDADKSPEKKLVVDASLISSPSNPGHAEIHGNIVIAGEPYHMKLILTATNLLEYMEGENGFKLLLTTPSQKTIVLGASCDVQLEGTTTKVVSVIEYKNMENKEYKYTSVIALEKLGGPYNYVVKAKVIYKQPETQEIMLETEVKHQWTPEEHLVAFKVGAKAPVLKMPLMIAFSIHNTRGSFVGFCKIERNTPSNVFEWKIQMTPEGGIEVVEAGLDIKAINEVLKIVHAVVTFEEEGYQAYGQETAKYQYRFTRPSPTTYIMQMRTPTRTIEGRAKLSPRESGIKFYPNKGKAEAKYEVGYKANHQGSWGQHASNIEVRMNHPTLPKPIMVAAHYTAIGETIKGTIELDIFPEEENKITGTLETQRISENAIRVEVFLTGKILQVNPKAIVTVAYAPETFALDVVFHKTPSTAPVFALAAKYDKTSAHNAAATFTVEMEQRPVFEITAVAEPEEEVPCNGIRIKAIANAPAFGKYNIFSKMCKPAFIELTTMRHGGEKEYTARLGLRYPDTAEAGVYVASGRAEEIRGVAVAAVKLASPTMLKVEMAYGPEEAQVLMNEMTEEYEKAAVLFKSVVMEVVHFLEEEASAKGIHFPSSQLVTLLGVAKEEIEEIYRDILSDARIFDTEIIRDILASPVVSFVPRVYFGVWSEIVLLQHQLSVNIIQAIERFQGEFEGITEIIMEIVMEATRMAETGEVPKVLLDVLEQIRASKVFRIVKREVYEILEEYPEEYEAITHVVGNVMAMLERDVEIVRVGLMKMPAVQRIIDYIMNHFHSKQVFAVEAERVVSLILSELLYVSIEREGNGIEVQIPLHRPLYSLTQVAQEAVPNPITMLENLIFAYLEYIPIPVEHAIWAYYNFIPRYITDVLPPYPRTAMVVGGSEILTFDGLVVRAPRSPCKVLLAAHGSHRLMMSHPQPSAPPQLELNTPAASVVIKPDFEVLVNGRPLTGSQQTIGNIRIVNAAKHIEVGCPLMKVVVAKTGQVVAVEASGWTYGRVAGLLGPNTGEIADDRLMPTGVQASSPRELVSAWQEDQGCSTPEVPRSETTVARLIQCQTLLGIRSRCNPVVQPQPFINMCHAARNACDAAQAYRTICALRGVEEMRPWAC

SEQ ID NO: 12 - protein, 1603 aa, Caenorhabditis elegans
  nematode vitellogenin
MKSIIIASLVALAIAASPALDRTFSPKSEYVYKFDGLLLSGLPTRSSDASQTLISCRTRLQAVDDRYIHLQLTDIQYSASHIPQSEQWPKIESLEQRELSDEFKELLELPFRAQIRNGLISEIQFSSEDAEWSKNAKRSILNLFSLRKSAPVDEMNQDQKDMESDKDSVFFNVHEKTMEGDCRSRLHIVQEGEKTIYTKSVNFDKCITRPETAYGLRFGSECKECEKEGQFVKPQTVYTYTFKNEKLQESEVHSVYTLNVNGQEVVKSETRAKVTFVEESKINREIKKVSGPKEEIVYSMENEKLIEQFYQQGDKAEVNPFKAIEMEQKVEQLQEIFRQIQEHEQNTPETVHLIARAVRMFRMCTIEELKKVHTTIYTKAEKKVQLVIETSIAVAGTKNTIQHLIHHFEKKSITPLRAAELLKSVQETLYPSEHIADLLIQLAQSPLSEKYEPLRQSAWLAAGSVVRGFASKTQDLPLIRPASRQTKEKYVRVFMQHFRNADSTYEKVLALKTLGNAGIDLSVYELVQIIQDPRQPLSIRTEAVDALRLLKDVMPRKIQKVLLPVYKNRQNKPELRMAALWRMMHTIPEEPVLAHIVSQMENESNQHVAAFTYHVLRQFYKSTNPCYQQLAVRCSKILLFTRYQPQEQMLSTYSQLPLFNSEWLSGVQFDFATIFEKNAFLPKEVQASLETVFGGNWNKYFAQVGFSQQNFEQVILKTLEKLSLYGKQSDELRSRRVQSGIQMLQEIVKKMNIRPRVQQTDSQNAHAVFYLRYKEMDYIVLPIDMETIDTLVEKYVRNGEFDIKSLLTFLTNDSKFELHRALFFYEAERRIPTTIGMPLTISGKMPTILSINGKVSIELEKLGARLVLDIVPTVATTHVTEMPLLYPVIEQGVKSLQSARLHTPLRFESTVELKKNTLEITHKFVVPENKKTTVSVHTRPVAFIRVPKNQDSEYVEAEEKTISHSQYQMSTEEIDRQYETFGLRINAQGNVLSQWTLPMVLMTEQDFEYTLENKNRPVEFTARVTIGNLEKTDLSEIKFDKIFEKEFDLENNESENRRQYFHKMIREIQSEQGFKNLITLKLEAPQQMYWNTELRTVCDKWIRMCKVEMDARRSPMEHENKEWTLRTELLAARPQMPSSLRQLREQPHREVQLAFNAKWGSSKKSEITVNAQLEQSTEQKKFIRNIEREYKGIPEYELLIKAARLNQVNVVSEYKLTPQSEYTFSRIFDLIKAYNFWTVSEKRVQNENRRVVLQLSVEPLSRQSHEHDHQTPEQEVELKNARIPRVVLPTIARSAMFQQTWEKTGATCKVDQSEVSTFHNVIYRAPLTTCYSLVAKDCSEQPRFAVLAKKINKNSEELLVKVVRREEEIVVKKSDDKFLVKVDGKKVNPTELEQYNIEILGDNLIVIRLPQGEVRFDGYTVKTNMPSVASQNQLCGLCGNNDGERDNEFMTADNYETEDVEEFHRSYLLKNEECEFEFDRISEKKNYRNKWNREEKKSDYESSSDYESNYDEKETEEELVKKTLIKEFSNRVCFSIEPVSECRRGLESEKTSNKKIRFTCMPRHRQERSRFLQGSSEQTVAELVDFPVSFVESVKIPTACVAY

SEQ ID NO: 13 - protein, 1660 aa, Oscheius tipulae
  rhabditid nematode vitellogenin
MRFAVLLALFGLALAARQSSVSEQYYRSGREYRYQFNGHLSAGLPIPGEENSATRIQSLIRIQPENGDFMRLQMTKTRFATSEEDRVLSFENMNEVPVSEKVEKVLSLPIRFSYRHGMVGEIEFSTEEQTWSSNIKKAVVNMLQVNLVKKGMSEKNEYETEHDNDFFLSNERTLEGECEVAYTKIEKSEKEQQWTKSINFEKCSLRPEIVYGRRYAEECNECRERDEKFSSTVFFYNITGTPEFLINSVELQSKHMFAPVTEQKQLITARITNRLELVYSGEQKEQIEAVRNSDKKENLLYNPEHEIAEEKFAQTGDEEVSSSYSQLRRSGRNHPSAAEPSLQADRANRAREQPRPRKILLVNYQPCIQARLVKSLRFATEDNLSSIRSLVSQKSEVVQSLYWDALAIAGTKVTVSHLLEKINNKEISPMKASQLMKILAEVRIPSEQIAQELHRFCESDIVSRSAVLRQSCWLSYGSVLNGVCGQTKNVYGSEITETRKQCTRQMKEKYIRELIEKMNQAESRYEKVLFVKAIANAGIDTSVVELEKIIRNQEVEKTVRMQAIDALRRLRLSMPKKIQNVLMPVYRNHKETPGIRISALHMIMQTQPTSGVLDMIVRGLEKERSQQVRVYTWSTLKTLSESENPAEKEIRRRVSQSLASIPVEEQKYLESKHKTFNWFNMQSGATLNWATIFSNDSVLPKEITASLETVFGGEWNKYLAQIGLYQNNLDSVLSKLLQKVEETGLEQLVVRGKRSSSFFRPAEMLRSLVESLRISHRQVPAMSPIAMIYLRYKDQDYAFLPIDIDTLPEMIRRVARDGQLDLSEIEKVLTQAARFTVAGSAFFHETVRKVPSALGMPQVMTSKMPTVAQMNGEVKFDLEPLNSDKFTGLRLRVKAEPHVASTHVCKLELFTPIGGQGIKLLHGGRIQAPIDSEIEINWEKKLIVKATIKSPEQKRHIAHFMTRPVLFSREVTMDKQMRQYPEPREKTIQLRENRFPIHAFERQYFEQTGLKMTVSGHYRRPFTTAFTLGESIMMSSSSLPRMLSLKEVVAYMSFSGFEETEMDEPRLLNRFYEKETELFETEKNVEYEQEDKEPKSSQLQSQIRKVKSEGKAYKHRVHMKIHTVGGPKTQEAECEIRALCDERVRFCRLNLDASRSPIQGESRQWQLKSSAEWLYPEVPSTMKKMLESRREWNAMWTGKWGSEKQNEVTIRVQGEQSSEQKFWMKKAEREQSPLTSGGQASRAAQLNQYNIHATYEVTPETEFWMENVYSMFKTYYFFSAEVQPKQNKENRIQCQITLEPFTRQLFNVTVMSPKEKLVLELENQQTPFRLPAVNIGREFGRVQSVRHVVKAVERQTRPECIVKSKEIQTFDEVFYRTPVMECFSVLAKDCSENPDFAVLMRKVSKRGEEKMWKVISRENVIELEKKSEEMSVRVNGKEISEDKWEDFGISQRGEEKFFIDAEKVTVEFDGFQAKIQMSSLYKNKQCGLCGHYDGEKTNEFRRADNEETDDIEEFSRSYLSKDDECEVDEQEMTNKRNYKVLREETSSSEEISESLIELYSEAFQSREAHHRLGRREDRQVCFSQQAWNKCLKSKDNKTETKNVHFKCLTETTRSPRISSVFSQADISENLSDVEGLPSLAEPSPHSRFLPCVIPSLSLSQ

SEQ ID NO: 14 - protein, 1687 aa, Fundulus heteroclitus
  killifish vitellogenin II precursor (Vtg II), yolk protein precursor
  SIGNAL (1)..(15): signal peptide
  PEPTIDE (16)..(1687): mature chain
MRVLVLALTVALVAGNQVSYAPEFAPGKTYEYKYEGYILGGLPEEGLAKAGVKIQSKVLIGAAGPDSYILKLEDPVISGYSGIWPKEVFHPATKLTSALSAQLLTPVKFEYANGVIGKVFAPPGISTNVLNVFRGLLNMFQMNIKKTQNVYDLQETGVKGVCKTHYILHEDSKADRLHLTKTTDLNHCTDSIHMDVGMAGYTEKCAECMARGKTLSGAISVNYIMKPSASGTLILEATATELLQYSPVNIVNGAVQMEAKQTVTFVDIRKTPLEPLKADYIPRGSLKYELGTEFLQTPIQLLRITNVEAQIVESLNNLVSLNMGHAHEDSPLKFIELIQLLRVAKYESIEALWSQFKTKIDHRHWLLSSIPAIGTHVALKFIKEKIVAGEVTAAEAAQAIMSSTHLVKADLEAIKLQEGLAVTPNIRENAGLRELVMLGFGIMVHKYCVENPSCPSELVRPVHDIIAKALEKRDNDELSLALKVLGNAGHPSSLKPIMKLLPGFGSSASELELRVHIDATLALRKIGKREPKMIQDVALQLFMDRTLDPELRMVAVVVLFDTKLPMGLITTLAQSLLKEPNLQVLSFVYSYMKAFTKTTTPDHSTVAAACNVAIRILSPRFERLSYRYSRAFHYDHYHNPWMLGAAASAFYINDAATVLPKNIMAKARVYLSGVSVDVLEFGARAEGVQEALLKARDVPESADRLTKMKQALKALTEWRANPSRQPLGSLYVKVLGQDVAFANIDKEMVEKIIEFATGPEIRTRGKKALDALLSGYSMKYSKPMSAIEVRHIFPTSLGLPMELSLYTAAVTAASVEVQATISPPLPEDFHPAHLLKSDISMKASVTPSVSLHTYGVMGVNSPFIQASVLSRAKDHAALPKKMEARLDIVKGYFSYQFLPVEGVKTIASARLETVAIARDVEGLAAAKVTPVVPYEPIVSKNATLNLSQMSYYLNDSISASSELLPFSLQRQTGKNKIPKPIVKKMCATTYTYGIEGCVDIWSRNATFLRNTPIYAIIGNHSLLVNVTPAAGPSIERIEIEVQFGEQAAEKILKEVYLNEEEEVLEDKNVLMKLKKILSPGLKNSTKASSSSSGSSRSSRSRSSSSSSSSSSSSSSRSSSSSSRSSSSLRRNSKMLDLADPLNITSKRSSSSSSSSSSSSSSSSSSSSSSKTKWQLHERNFTKDHIHQHSVSKERLNSKSSASSFESIYNKITYLSNIVSPVVTVLVRAIRADHKNQGYQIAVYYDKLTTRVQIIVANLTEDDNWRICSDSMMLSHHKVMTRVTWGIGCKQYNTTIVAETGRVEKEPAVRVKLAWARLPTYIRDYARRVSRYISRVAEDNGVNRTKVASKPKEIKLTVAVANETSLNVTLNTPKNTFFKLGWVLPFYLPINNTAAELQAFQGRWMDQVTYMLTKSAAAECTVVEDTVVTFNNRKYKTETPHSCHQVLAQDCTSEIKFIVLLKRDQTAERNEISIKIENIDVDMYPKDNAVVVKVNGVEIPLTNLPYQHPTGNIQIRQREEGISLHAPSHGLQEVFLSLNKVQVKVVDWMRGQTCGLCGKADGEVRQEYSTPNERVSRNATSFAHSWVLPAKSCRDASECYMQLESVKLEKQISLEGEESKCYSVEPVWRCLPGCAPVRTTSVTVGLPCVSLDSNLNRSDSLSSIYQKSVDVSETAESHLACRCTPQCA

SEQ ID NO: 15 - protein, 1659 aa, Oncorhynchus mykiss
  rainbow trout vitellogenin
MRAVVLALTLALVASQSVNFAPDFAASKTYVYKYEALLLGGLPEEGLARAGVKVISKVLISAVAENTYLLKLVNPEIFEYSGVWPKDPFVPAAKLTSALAAQFSIPIKFEYAKGVVGKVLAPTAVSETVLNVHRGILNILQLNIKKTQNVYELQEAGAQGVCKTHYVIREDAKAERIHLTKSKDLNNCQQRIMKDFGLAYTEKCVECRQRGEALMGAATYNYLMKPADNGALILEATVTELHQFTPFNEMSGAAQMEAKQMLTFVEIKKDPIIVPDNNYVHRGSIRYEFATEILQMPIQLLKISNARAQAVKILNHLVTYNTAPVHEDAPLKFLQFIQLLRMASSETINAIWAEFKAKPAYRHWILDAVPSIGSSVAVRFIKEKFLAGDITIFEAAQALVAAVHMVAADLETVKLVESLAFNHKIQTHPVLRELTMLGYGTMVSKYCVEHPNCPAELVKPIHELAVQAVANSKFEELSMVLKALGNAGHPASIKPITKLLPVFGTAAAALPLRVQADAVLALRNIAKREPRMVQEVAVQLFMDKALHPELRMLACIVLFETKPPMGLVITLASILKTEKNMQVASFTYSHMMSLTRSTAPDFASVAAACNVAVKMLSNKFRRLSCHFSQAIHLDAYSNPLRIGAAASAFYINDAATLFPRTVVAKARTYFAGAAADVLEVGVRTEGIQEALLKLPPAPENADRITKMRRVIKALSDWRSLATSKPLASIYVKFFGQEIAFANIDKSIIDQALQLANSPSAHALGRNALKALLAGATFQYVKPLLAAEVRRIFPTAVGLPMELSYYTAAVAKAYVNVRATLTPALPETFHAAQLLKTNIELHAEVRPSIVMHTFAVMGVNTAFIQAAIMARAKVRTIVPAKFAAQLDIANGNFKFEAFPVSPPEHIAAAHIETFAVARNVEDVPAERITPLIPAQGVARSTQQSRDKLTSMIADSAASFAGSLSRSSEILYSDLPSNFKPIIKAIVVHLEETICVERLGVKACFEFTSESAAFIRNTLFYNMIGKHSVLISVKPSASEPAIERLEFEVQVGPKAAEKIIKVITMNEEEEAPEGKTVLLKLKKILLPDLKNGTRASSSSSSSSSSSSRSSSSRSRSRKSESSSSSSSSSSRISKRDGPDQPYNPNDRKFKKNHKDSQSTSNVISRSKSSASSFHAIYKQDKFLGNKLAPMVIILFRLVRADHKIEGYQVTAYLNKATSRLQIIMAALDENDNWKLCADGVLLSKHKVTAKIAWGAECKDYNTFITAETGLVGPSPAVRLRLSWDKLPKVPKAVWRYVRIVSEFIPGYIPYYLADLVPMQKDKNNEKQIQFTVVATSERTLDVILKTPKMTLYKLGVNLPCSLPFESMTDLSPFDDNIVNKIHYLFSEVNAVKCSMVRDTLTTFNNKKYKINMPLSCYQVLAQDCTTELKFMVLLKKDHASEQNHINVKISDIDVDLYTEDHGVIVKVNEMEISNDNLPYKDPSGSIKIDRKGKGVSLYAPSHGLQEVYFDKYSWKIKVVDWMKGQTCGLCGKADGENRQEYRTPSGRLTKSSVSFAHSWVLPSDSCRDASECLMKLESVKLEKQVIVDDRESKCYSVEPVLRCLPGCLPVRTTPITIGFHCLPVDSNLNRSEGLSSIYEKSVDLMEKAEAHVACRCSEQCM

SEQ ID NO: 16 - protein, 1704 aa, Fundulus heteroclitus
  killifish vitellogenin 1 precursor (Vtg I), yolk protein precursor
MKAVVLALTLAFVAGQNFAPEFAAGKTYVYKYEALILGGLPEEGLARAGLKISTKLLLSAADQNTYMLKLVEPELSEYSGIWPKDPAVPATKLTAALHLSSQFPSSLNTPMVFVGKVFAPEEVSTLVLNIYRGILNILQLNIKKTHKVYDLQEVGTQGVCKTLYSISEDARIENILLTKTRDLSNCQERLNKDIGLAYTEKCDKCQEETKNLRGTTTLSYVLKPVADAVMILKAYVNELIQFSPFSEANGAAQMRTKQSLEFLEIEKEPIPSVKAEYRHRGSLKYEFSDELLQTPLQLIKISDAPAQVAEVLKHLATYNIEDVHENAPLKFLELVQLLRIARYEDLEMYWNQYKKMSPHRHWFLDTIPATGTFAGLRFIKEKFMAEEITIAEAAQAFITAVHMVTADPEVIKLFESLVDSDKVVENPLLREVVFLGYGTMVNKYCNKTVDCPVELIKPIQQRLSDAIAKNEEENIILYIKVLGNAGHPSSFKSLTKIMPIHGTAAVSLPMTIHVEAIMALRNIAKKESRMVQELALQLYMDKALHPELRMLSCIVLFETSPSMGLVTTVANSVKTEENLQVASFTYSHMKSLSRSPATIHPDVAAACSAAMKILGTKLDRLSLRYSKAVHVDLYNSSLAVGAAATAFYINDAATFMPKSFVAKTKGFIAGSTAEVLEIGANIEGLQELILKNPALSESTDRITKMKRVIKALSEWRSLPTSKPLASVYVKFFGQEIGFANIDKPMIDKAVKFGKELPIQEYGREALKALLLSGINFHYAKPVLAAEMRRILPTVAGIPMELSLYSAAVAAASVEIKPNTSPRLSADFDVKTLLETDVELKAEIRPMVAMDTYAVMGLNTDIFQAALVARAKLHSVVPAKIAARLNIKEGDFKLEALPVDVPENITSMNVTTFAVARNIEEPLVERITPLLPTKVLVPIPIRRHTSKLDPTRNSMLDSSELLPMEEEDVEPIPEYKFRRFAKKYCAKHIGVGLKACFKFASQNGASIQDIVLYKLAGSHNFSFSVTPIEGEVVERLEMEVKVGAKAAEKLVKRINLSEDEETEEGGPVLVKLNKILSSRRNSSSSSSSSSSSSSESRSSRSSSSSSSSSRSSRKIDLAARTNSSSSSSSRRSRSSSSSSSSSSSSSSSSSSSSRRSSSSSSSSSSSSSRSSRRVNSTRSSSSSSRTSSASSLASFFSDSSSSSSSSDRRSKEVMEKFQRLHKKMVASGSSASSVEAIYKEKKYLGEEEAVVAVILRAVKADKRMVGYQLGFYLDKPNARVQIIVANISSDSNWRICADAVVLSKHKVTTKISWGEQCRKYSTNVTGETGIVSSSPAARLRVSWERLPSTLKRYGKMVNKYVPVKILSDLIHTKRENSTRNISVIAVATSEKTIDIITKTPMSSVYNVTMHLPMCIPIDEIKGLSPFDEVIDKIHFMVSKAAAAECSFVEDTLYTFNNRSYKNKMPSSCYQVAAQDCTDELKFMVLLRKDSSEQHHINVKISEIDIDMFPKDDNVTVKVNEMEIPPPACLTATQQLPLKIKTKRRGLAVYAPSHGLQEVYFDRKTWRIKVADWMKGKTCGLCGKADGEIRQEYHTPNGRVAKNSISFAHSWILPAESCRDASECRLKLESVQLEKQLTIHGEDSTCFSVEPVPRCLPGCLPVKTTPVTVGFSCLASDPQTSVYDRSVDLRQTTQAHLACSCNTKCS

SEQ ID NO: 17 - protein, 1677 aa, Acipenser transmontanus
  white sturgeon vitellogenin, egg yolk protein precursor
  SIGNAL (1)..(8): signal peptide
  PEPTIDE (9)..(1677): mature peptide
  PEPTIDE (9)..(1081): lipovitellin I
  PEPTIDE (1097)..(1273): phosvitin
  PEPTIDE (1274)..(1677): lipovitellin II
LTIALVGSQQTKYEPSFSGSKTYQYKYEGVILTGLPEKGLARAGLKVHCKVEISEVAQKTYLLKILNPEIQEYNGIWPKAPFYPASKLTQALASQLTQPIKFQYRNGQVGDIFASEDVSDTVLNIQRGILNMLQLTIKTTQNVYGLQENGIAGICEASYVIQEDRKANKIIVTKSKDLNNCNEKIKMDIGMAYSHTCSNCRKIRKNTRGTAAYTYILKPTDTGTLITQATSQEVHQLTPFNEMTGAAITEARQKLVLEDAKVIHVTVPEQELKNRGSIQYQFASEILQTPIQLFKTRSPETKIKEVLQHLVQNNQQQVQSDAPSKFLQLTQLLRACTHENIEGIWRQYEKTQLYRRWILDALPAAATPTAFRFITQRIMKRDLTDAEAIQTLVTAMHLVQTNHQIVQMAAELVFDRANLKCPVLRKHAVLAYGSMVNRYCAETLNCREEALKPLHDFANDAISRAHEEETVLALKALGNAGQPSSIKRIQKCLPGFSSGASQLPVKIQVDAVMALRNIAKKEPGKVQELTMQLFMDHQLHSEVRMVASMVLLETRPSMALVATLAEALLKETSLQVASFTYSHMKAITRSTAPENHALSSACNVAVKLLSRKLDRLSYRYSKAMHMDTFKYPLMAGAAANIHIINNAASILPSAVVMKFQAYILSATADPLEIGLHTEGLQEVLMQNHEHIDQMPSAGKIQQIMKMLSGWKSVPSEKTLASAYIKLFGQEISFSRLDKKTIQEALQAVREPVERQTVIKRVVNQLERGAAAQLSKPLLVAEVRRILPTCIGLPMEMSLYVSAVTTADINVQAHITPSPTNDFNVAQLLNSNIVLHTDVTPSIAMHTIAVMGINTHVIQTGVELHVKARTTVPMKFTAKIDLKEKNFKIESEPCQQETEVLSLSAQAFAISRNVEDLDAAKKNPLLPEEAVRNILNEQFNSGTEDSNERERAGKFARPSAEMMSQELMNSGEHQNRKGAHATRSACAKAKNFGFEVCFEGKSENVAFLRDSPLYKIIGQHHCKIALKPSHSSEATIEKIQLELQTGNKAASKIIRVVAMQSLAEADEMKGNILKKLNKLLTVDGETQDSTLRGFKRRSSSSSSSSSSSSSSSSSSSSSSSQQSRMEKRMEQDKLTENLERDRDHMRGKQSKNKKQEWKNKQKKHHKQLPSSSSSSSSSSSGSNSSSSSSSSSSSSSRSHNHRNNTRTLSKSKRYQNNNNSSSSSGSSSSSEEIQKNPEIFAYRFRSHRDKLGFQNKRGRMSSSSSSSSSSSSQSTLNSKQDAKFLGDSSPPIFAFVARAVRSDGLQQGYQVAAYTDNRVSRPRVQLLATEIIEKSRWQICADAILASNYKAMALMRWGEECQDYKVAVSAVTGRLASHPSLQIKAKWSRIPRAAKQTQNILAEYVPGAAFMLGFSQKEQRNPSKQFKIILAVTSPNTIDTLIKAPKITLFKQAVQIPVQIPMEPSDAERRSPGLASIMNEIPFLIEEATKSKCVAQENKFITFDGVKFSYQMPGGCYHILAQDCRSKVRFMVMLKQASMSKNLRAVNAKIYNKDIDILPTTKGSVRLLINNNEIPLSQLPFTDSSGNIHIKRADEGVSVSAQQYGLESLYFDGKTVQVKVTSEMRGKTCGLCGHNDGERRKEFRMPDGRQARGPSVSPTPGLCLEKTATEAASFCVIM

SEQ ID NO: 18 - protein, 1807 aa, Xenopus laevis
  African clawed frog vitellogenin
MKGIVLALLLALAGSERTHIEPVFSESKISVYNYEAVILNGFPESGLSRAGIKINCKVEISAYAQRSYFLKIQSPEIKEYNGVWPKDPFTRSSKLTQALAEQLTKPARFEYSNGRVGDIFVADDVSDTVANIYRGILNLLQVTIKKSQDVYDLQESSVGGICHTRYVIQEDKRGDQIRIIKSTDFNNCQDKVSKTIGLELAEFCHSCKQLNRVIQGAATYTYKLKGRDQGTVIMEVTARQVLQVTPFAERHGAATMESRQVLAWVGSKSGQLTPPQIQLKNRGNLHYQFASELHQMPIHLMKTKSPEAQAVEVLQHLVQDTQQHIREDAPAKFLQLVQLLRASNFENLQALWKQFAQRTQYRRCLLDALPMAGTVDCLKFIKQLIHNEELTTQEAAVLITFAMRSARPGQRNFQISADLVQDSKVQKYSTVHKAAILAYGTMVRRYCDQLSSCPEHALEPLHELAAEAANKGHYEDIALALKALGNAGQPESIKRIQKFLPGFSSSADQLPVRIQTDAVMALRNIAKEDPRKVQEILLQIFMDRDVRTEVRMMACLALFETRPGLATVTAIANVAARESKTNLQLASFTFSQMKALSKSSVPHLEPLAAACSVALKILNPSLDNLGYRYSKVMRVDTFKYNLMAGAAAKVFIMNSANTMFPVFILAKFREYTSLVENDDIEIGIRGEGIEEFLRKQNIQFANFPMRKKISQIVKSLLGFKGLPSQVPLISGYIKLFGQEIAFTELNKEVIQNTIQALNQPAERHTMIRNVLNKLLNGVVGQYARRWMTWEYRHIIPTTVGLPAELSLYQSAIVHAAVNSDVKVKPTPSGDFSAAQLLESQIQLNGEVKPSVLVHTVATMGINSPLFQAGIEFHGKVHAHLPAKFTAFLDMKDRNFKIETPPFQQENHLVEIRAQTFAFTRNIADLDSARKTLVVPRNNEQNILKKHFETTGRTSAEGASMMEDSSEMGPKKYSAEPGHHQYAPNINSYDACTKFSKAGVHLCIQCKTHNAASRRNTIFYQAVGEHDFKLTMKPAHTEGAIEKLQLEITAGPKAASKIMGLVEVEGTEGEPMDETAVTKRLKMILGIDESRKDTNETALYRSKQKKKNKIHNRRLDAEVVEARKQQSSLSSSSSSSSSSSSSSSSSSSSSSSSSPSSSSSSSYSKRSKRREHNPHHQRESSSSSSQEQNKKRNLQENRKHGQKGMSSSSSSSSSSSSSSSSSSSSSSSSSSSSEENRPHKNRQHDNKQAKMQSNQHQQKKNKFSESSSSSSSSSSSEMWNKKKHHRNFYDLNFRRTARTKGTEHRGSRLSSSSESSSSSSESAYRHKAKFLGDKEPPVLVVTFKAVRNDNTKQGYQMVVYQEYHSSKQQIQAYVMDISKTRWAACFDAVVVNPHEAQASLKWGQNCQDYKINMKAETGNFGNQPALRVTANWPKIPSKWKSTGKVVGEYVPGAMYMMGFQGEYKRNSQRQVKLVFALSSPRTCDVVIRIPRLTVYYRALRLPVPIPVGHHAKENVLQTPTWNIFAEAPKLIMDSIQGECKVAQDQITTFNGVDLASALPENCYNVLAQDCSPEMKFMVLMRNSKESPNHKDINVKLGEYDIDMYYSADAFKMKINNLEVSEEHLPYKSFNYPTVEIKKKGNGVSLSASEYGIDSLDYDGLTFKFRPTIWMKGKTCGICGHNDDESEKELQMPDGSVAKDQMRFIHSWILPAESCSEGCNLKHTLVKLEKAIATDGAKAKCYSVQPVLRCAKGCSPVKTVEVSTGFHCLPSDVSLDLPEGQIRLEKSEDFSEKVEAHTACSCETSPCAA

SEQ ID NO: 19 - protein, 1852 aa, Gallus gallus
  chicken vitellogenin
MRGIILALVLTLVGSQKFDIDPGFNSRRSYLYNYEGSMLNGLQDRSLGKAGVRLSSKLEISGLPENAYLLKVRSPQVEEYNGVWPRDPFTRSSKITQVISSCFTRLFKFEYSSGRIGNIYAPEDCPDLCVNIVRGILNMFQMTIKKSQNVYELQEAGIGGICHARYVIQEDRKNSRIYVTRTVDLNNCQEKVQKSIGMAYIYPCPVDVMKERLTKGTTAFSYKLKQSDSGTLITDVSSRQVYQISPFNEPTGVAVMEARQQLTLVEVRSERGSAPDVPMQNYGSLRYRFPAVLPQMPLQLIKTKNPEQRIVETLQHIVLNNQQDFHDDVSYRFLEVVQLCRIANADNLESIWRQVSDKPRYRRWLLSAVSASGTTETLKFLKNRIRNDDLNYIQTLLTVSLTLHLLQADEHTLPIAADLMTSSRIQKNPVLQQVACLGYSSVVNRYCSQTSACPKEALQPIHDLADEAISRGREDKMKLALKCIGNMGEPASLKRILKFLPISSSSAADIPVHIQIDAITALKKIAWKDPKTVQGYLIQILADQSLPPEVRMMACAVIFETRPALALITTIANVAMKESKTNMQVASFVYSHMKSLSKSRLPFMYNISSACNIALKLLSPKLDSMSYRYSKVIRADTYFDNYRVGATGEIFVVNSPRTMFPSAIISKLMANSAGSVADLVEVGIRVEGLADVIMKRNIPFAEYPTYKQIKELGKALQGWKELPTETPLVSAYLKILGQEVAFININKELLQQVMKTVVEPADRNAAIKRIANQILNSIAGQWTQPVWMGELRYVVPSCLGLPLEYGSYTTALARAAVSVEGKMTPPLTGDFRLSQLLESTMQIRSDLKPSLYVHTVATMGVNTEYFQHAVEIQGEVQTRMPMKFDAKIDVKLKNLKIETNPCREETEIVVGRHKAFAVSRNIGELGVEKRTSILPEDAPLDVTEEPFQTSERASREHFAMQGPDSMPRKQSHSSREDLRRSTGKRAHKRDICLKMHHIGCQLCFSRRSRDASFIQNTYLHKLIGEHEAKIVLMPVHTDADIDKIQLEIQAGSRAAARIITEVNPESEEEDESSPYEDIQAKLKRILGIDSMFKVANKTRHPKNRPSKKGNTVLAEFGTEPDAKTSSSSSSASSTATSSASSSASSPNRKKPMDEEENDQVKQARNKDASSSSRSSKSSNSSKRSSSKSSNSSKRSSSSSSSSSSSSRSSSSSSSSSSNSKSSSSSSKSSSSSSRSRSSSKSSSSSSSSSSSSSSKSSSSRSSSSSSKSSSHHSHSHHSGHLNGSSSSSSSSRSVSHHSHEHHSGHLEDDSSSSSSSSVLSKIWGRHEIYQYRFRSAHRQEFPKRKLPGDRATSRYSSTRSSHDTSRAASWPKFLGDIKTPVLAAFLHGISNNKKTGGLQLVVYADTDSVRPRVQVFVTNLTDSSKWKLCADASVRNAPQAVAYVKWGWDCRDYKVSTELVTGRFAGHPAAQVKLEWPKVPSNVRSVVEWFYEFVPGAAFMLGFSERMDKNPSRQARMVVALTSPRTCDVVVKLPDIILYQKAVRLPLSLPVGPRIPASELQPPIWNVFAEAPSAVLENLKARCSVSYNKIKTFNEVKFNYSMPANCYHILVQDCSSELKFLVMMKSAGEATNLKAINIKIGSHEIDMHPVNGQVKLLVDGAESPTANISLISAGASLWIHNENQGFALAAPGHGIDKLYFDGKTITIQVPLWMAGKTCGICGKYDAECEQEYRMPNGYLAKNAVSFGHSWILEEAPCRGACKLHRSFVKLEKTVQLAGVDSKCYSTEPVLRCAKGCSATKTTPVTVGFHCLPADSANSLTDKQMKYDQKSEDMQDTVDAHTTCSCENEECST

SEQ ID NO: 20 - protein, 1823 aa, Ichthyomyzon unicuspis
  lamprey vitellogenin precursor
  SIGNAL (1)..(14): signal peptide
  PEPTIDE (15)..(1823): mature chain
  PEPTIDE (15)..(707): lipovitellin LV-1N
  PEPTIDE (708)..(1074): lipovitellin LV-1C
  PEPTIDE (1306)..(1624): lipovitellin LV-2
MWKLLLVALAFALADAQFQPGKVYRYSYDAFSISGLPEPGVNRAGLSGEMKIEIHGHTHNQATLKITQVNLKYFLGPWPSDSFYPLTAGYDHFIQQLEVPVRFDYSAGRIGDIYAPPQVTDTAVNIVRGILNLFQLSLKKNQQTFELQETGVEGICQTTYVVQEGYRTNEMAVVKTKDLNNCDHKVYKTMGTAYAERCPTCQKMNKNLRSTAVYNYAIFDEPSGYIIKSAHSEEIQQLSVFDIKEGNVVIESRQKLILEGIQSAPAASQAASLQNRGGLMYKFPSSAITKMSSLFVTKGKNLESEIHTVLKHLVENNQLSVHEDAPAKFLRLTAFLRNVDAGVLQSIWHKLHQQKDYRRWILDAVPAMATSEALLFLKRTLASEQLTSAEATQIVYSTLSNQQATRESLSYARELLHTSFIRNRPILRKTAVLGYGSLVFRYCANTVSCPDELLQPLHDLLSQSSDRADEEEIVLALKALGNAGQPNSIKKIQRFLPGQGKSLDEYSTRVQAEAIMALRNIAKRDPRKVQEIVLPIFLNVAIKSELRIRSCIVFFESKPSVALVSMVAVRLRREPNLQVASFVYSQMRSLSRSSNPEFRDVAAACSVAIKMLGSKLDRLGCRYSKAVHVDTFNARTMAGVSADYFRINSPSGPLPRAVAAKIRGQGMGYASDIVEFGLRAEGLQELLYRGSQEQDAYGTALDRQTLLRSGQARSHVSSIHDTLRKLSDWKSVPEERPLASGYVKVHGQEVVFAELDKKMMQRISQLWHSARSHHAAAQEQIRAVVSKLEQGMDVLLTKGYVVSEVRYMQPVCIGIPMDLNLLVSGVTTNRANLHASFSQSLPADMKLADLLATNIELRVAATTSMSQHAVAIMGLTTDLAKAGMQTHYKTSAGLGVNGKIEMNARESNFKASLKPFQQKTVVVLSTMESIVFVRDPSGSRILPVLPPKMTLDKGLISQQQQQPHHQQQPHQHGQDQARAAYQRPWASHEFSPAEQKQIHDIMTARPVMRRKQHCSKSAALSSKVCFSARLRNAAFIRNALLYKITGDYVSKVYVQPTSSKAQIQKVELELQAGPQAAEKVIRMVELVAKASKKSKKNSTITEEGVGETIISQLKKILSSDKDKDAKKPPGSSSSSSSSSSSSSSSSSSDKSGKKTPRQGSTVNLAAKRASKKQRGKDSSSSSSSSSSSSDSSKSPHKHGGAKRQHAGHGAPHLGPQSHSSSSSSSSSSSSSSASKSFSTVKPPMTRKPRPARSSSSSSSSDSSSSSSSSSSSSSSSSSSSSSSSESKSLEWLAVKDVNQSAFYNFKYVPQRKPQTSRRHTPASSSSSSSSSSSSSSSSSSSDSDMTVSAESFEKHSKPKVVIVLRAVRADGKQQGLQTTLYYGLTSNGLPKAKIVAVELSDLSVWKLCAKFRLSAHMKAKAAIGWGKNCQQYRAMLEASTGNLQSHPAARVDIKWGRLPSSLQRAKNALLENKAPVIASKLEMEIMPKKNQKHQVSVILAAMTPRRMNIIVKLPKVTYFQQGILLPFTFPSPRFWDRPEGSQSDSLPAQIASAFSGIVQDPVASACELNEQSLTTFNGAFFNYDMPESCYHVLAQECSSRPPFIVLIKLDSERRISLELQLDDKKVKIVSRNDIRVDGEKVPLRRLSQKNQYGFLVLDAGVHLLLKYKDLRVSFNSSSVQVWVPSSLKGQTCGLCGRNDDELVTEMRMPNLEVAKDFTSFAHSWIAPDETCGGACALSRQTVHKESTSVISGSRENCYSTEPIMRCPATCSASRSVPVSVAMHCLPAESEAISLAMSEGRPFSLSGKSEDLVTEMEAHVSCVA

SEQ ID NO: 21 - DNA, 30 bp, Artificial Sequence
  Description of Artificial SequencePCR mutagenesis primer to introduce ApaI restriction site
ccatgggcccagcacactgaggagtgcagc

SEQ ID NO: 22 - DNA, 43 bp, Artificial Sequence
  Description of Artificial Sequencemutation primer to create SpeI restriction site and new internal start codon
tcttgctgtggctctactagtgatggaccagtccaacttggcc

SEQ ID NO: 23 - DNA, 43 bp, Artificial Sequence
  Description of Artificial Sequencemutation primer to create SpeI restriction site and new internal start codon
ccaagttggactggtccatcactagtagagccacagcaagagc

SEQ ID NO: 24 - protein, 6 aa, Artificial Sequence
  Description of Artificial Sequence6xHis C-terminal tag
HHHHHH